SEQUENCE LISTING (230 sequences)

SEQ ID NO: 1 - protein, 439 aa, Plasmodium falciparum
MCMGRMISIINIILFYFFLWVKKSISELLSSTQYVCDFYFNPLTNVKPTVVGSSEIYEEVGCTINNPTLGDHIVLICPKKNNGDFSNIEIVPTNCFESHLYSAYKNDSSAYHLEKLDIDKKYAINSSFSDFYLKILVIPNEYKSHKTIYCRCDNSKTEKNIPGQDKILKGKLGLVKIILRNQYNNIIELEKTKPIIHNKKDTYKYDIKLKESDILMFYMKEETIVESGNCEEILNTKINLLSNNNVVIKMPSIFINNINCMLSSQDQNNEKNYINLKADKTKHIDGCDFTKPKGKGIYKNGFIINDIPNEEERICTVHLWNKKNQTIAGIKCPYKLIPPYCFKHVLYEKEIDSQKTYKTFLLSDVLDTPNIEYYGNNKEGMYMLALPTKPEKTNKIRCICEQGGKKAVMELHIASTSTKYISMFLIFFLIVIFYMYVSI

SEQ ID NO: 2 - protein, 433 aa, Plasmodium vivax
MKLLTFAAATYGFLLKECLNSFIFPTKHLCDFALNPHSSIKPVLKEASGKDEEVWCSVHNPSLTDYVAMVCPKKKGGDYTELETVPANCFTKHLYSPYDSEENEKDMELLELDPKLSFNRTFNDFVLKVLVIPGYYKHNKTIYCRCDNRKTKKGEDQEKIEEGKVGLVKIVLNKKEKKPRGIDFTETDELEQTDIVQNGNDKLVKVKENETIHFKFNSNQKLEIKECENVINMKYGFLQEHVLNFRFPAVFLSSENCTITVIESAKTPVRIIIKTQKTENIDGCDFTKPSGEGDYQDGFALEELKSNEKICTIHIGSSKKKISAGIKCPYKLTPTYCFRHVLYEKDVNGVKSYHPFLLTDVLGTLDVEFYSNAQEGSYIIGLPTNPQKYSVVRCVCEHNGKAGIMELRIASSSGWAFLSLTLLLLLIALLSAC

SEQ ID NO: 3 - DNA, 21 bp, Artificial Sequence
  Primer
agagatactatgattatttta

SEQ ID NO: 4 - DNA, 20 bp, Artificial Sequence
  Primer
tgtcattatgaatggattcc

SEQ ID NO: 5 - DNA, 22 bp, Artificial Sequence
  Primer
gaaacttctacgggtttctgta

SEQ ID NO: 6 - DNA, 22 bp, Artificial Sequence
  Primer
gaaattatacacacacgcaaac

SEQ ID NO: 7 - DNA, 26 bp, Artificial Sequence
  Primer
gacaagtatttctatttgttagatca

SEQ ID NO: 8 - DNA, 21 bp, Artificial Sequence
  Primer
tcgttatatcaacaattgcat

SEQ ID NO: 9 - DNA, 25 bp, Artificial Sequence
  Primer
ctttgcttacgttttctttaaattc

SEQ ID NO: 10 - DNA, 26 bp, Artificial Sequence
  Primer
tcaagaaaccttacaacatgataaga

SEQ ID NO: 11 - DNA, 27 bp, Artificial Sequence
  Primer
ttcttatcatgttgtaaggtttcttga

SEQ ID NO: 12 - DNA, 26 bp, Artificial Sequence
  Primer
tttatatttaacccttcccaattttt

SEQ ID NO: 13 - DNA, 23 bp, Artificial Sequence
  Primer
tttcatttttgataaaggataag

SEQ ID NO: 14 - DNA, 22 bp, Artificial Sequence
  Primer
atatttaacccttcccaatttt

SEQ ID NO: 15 - DNA, 24 bp, Artificial Sequence
  Primer
aaagcagaataatatgtacgatca

SEQ ID NO: 16 - DNA, 22 bp, Artificial Sequence
  Primer
ggtggtggtagtagtagtggtt

SEQ ID NO: 17 - DNA, 23 bp, Artificial Sequence
  Primer
atcaataaaaatttaaccaagtg

SEQ ID NO: 18 - DNA, 22 bp, Artificial Sequence
  Primer
tgaaaaacatttttggagtgta

SEQ ID NO: 19 - DNA, 20 bp, Artificial Sequence
  Primer
ccagtttaccaagctttacg

SEQ ID NO: 20 - DNA, 21 bp, Artificial Sequence
  Primer
cagctattataaatggggatg

SEQ ID NO: 21 - DNA, 20 bp, Artificial Sequence
  Primer
gatgagagaaggttaaaata

SEQ ID NO: 22 - DNA, 20 bp, Artificial Sequence
  Primer
cttcaacacatctatggata

SEQ ID NO: 23 - DNA, 20 bp, Artificial Sequence
  Primer
gacgttcagattgtgtttcc

SEQ ID NO: 24 - DNA, 20 bp, Artificial Sequence
  Primer
gacaaaaacttaacgcaagc

SEQ ID NO: 25 - DNA, 22 bp, Artificial Sequence
  Primer
gacatgatgtgttctgttcatt

SEQ ID NO: 26 - DNA, 22 bp, Artificial Sequence
  Primer
ataatcccatgaaggataatca

SEQ ID NO: 27 - DNA, 20 bp, Artificial Sequence
  Primer
gcatcgatagggatttatga

SEQ ID NO: 28 - DNA, 23 bp, Artificial Sequence
  Primer
tctttgcaaataggaatattgtc

SEQ ID NO: 29 - DNA, 25 bp, Artificial Sequence
  Primer
tcatgtatttgtgaaaaagaagcaa

SEQ ID NO: 30 - DNA, 24 bp, Artificial Sequence
  Primer
ttttgaattaaggaaaacatcgac

SEQ ID NO: 31 - DNA, 20 bp, Artificial Sequence
  Primer
aacaaatgaaatgaagagca

SEQ ID NO: 32 - DNA, 20 bp, Artificial Sequence
  Primer
acatcaaggtgtccataaca

SEQ ID NO: 33 - DNA, 20 bp, Artificial Sequence
  Primer
tcttttgaagcagaaacgat

SEQ ID NO: 34 - DNA, 20 bp, Artificial Sequence
  Primer
cgaattcaagagttgcactt

SEQ ID NO: 35 - DNA, 20 bp, Artificial Sequence
  Primer
acatcaaaattttatgtatc

SEQ ID NO: 36 - DNA, 19 bp, Artificial Sequence
  Primer
ctctgtgctctcattgcac

SEQ ID NO: 37 - DNA, 34 bp, Artificial Sequence
  Primer
aaattaaacgatcacttattctgttgacaatgtt

SEQ ID NO: 38 - DNA, 19 bp, Artificial Sequence
  Primer
attgaagaacgtgtccaag

SEQ ID NO: 39 - DNA, 28 bp, Artificial Sequence
  Primer
tcaggagatatgttcgcaagaatcaaaa

SEQ ID NO: 40 - DNA, 22 bp, Artificial Sequence
  Primer
cttattgaagaacatgtccaag

SEQ ID NO: 41 - DNA, 28 bp, Artificial Sequence
  Primer
ttcccacgttgtaggatagtatacatca

SEQ ID NO: 42 - DNA, 18 bp, Artificial Sequence
  Primer
acctgaatctgtggaaag

SEQ ID NO: 43 - DNA, 25 bp, Artificial Sequence
  Primer
ctgcagcccaattaaatgaactaca

SEQ ID NO: 44 - DNA, 20 bp, Artificial Sequence
  Primer
aaacctgaatctttggaaag

SEQ ID NO: 45 - DNA, 27 bp, Artificial Sequence
  Primer
ctttggaactttcttctttgtcttgct

SEQ ID NO: 46 - DNA, 20 bp, Artificial Sequence
  Primer
aagttcatcttggcttaagt

SEQ ID NO: 47 - DNA, 29 bp, Artificial Sequence
  Primer
cctttcgtacctttcaatatagaggtgtt

SEQ ID NO: 48 - DNA, 20 bp, Artificial Sequence
  Primer
aagttcatcttggtttaagt

SEQ ID NO: 49 - DNA, 37 bp, Artificial Sequence
  Primer
caaaatgatgatcatcctgataatcatcataatgatg

SEQ ID NO: 50 - DNA, 24 bp, Artificial Sequence
  Primer
ctgttaatcatcataatgttaatt

SEQ ID NO: 51 - DNA, 25 bp, Artificial Sequence
  Primer
cttttctttgtgtgggatcgtttga

SEQ ID NO: 52 - DNA, 24 bp, Artificial Sequence
  Primer
ctgttaatcatcataattttaatt

SEQ ID NO: 53 - DNA, 38 bp, Artificial Sequence
  Primer
gggttattggtaagcatggaattaaaataaattttact

SEQ ID NO: 54 - DNA, 18 bp, Artificial Sequence
  Primer
ccacgttcttgccagaag

SEQ ID NO: 55 - DNA, 35 bp, Artificial Sequence
  Primer
cagtggtttcatgattaaaattatgttgtatagct

SEQ ID NO: 56 - DNA, 18 bp, Artificial Sequence
  Primer
ccacgttcttaccagaag

SEQ ID NO: 57 - DNA, 28 bp, Artificial Sequence
  Primer
ccttctctttgtttttagatgcatcact

SEQ ID NO: 58 - DNA, 16 bp, Artificial Sequence
  Primer
caatgcaagagaaatt

SEQ ID NO: 59 - DNA, 29 bp, Artificial Sequence
  Primer
gcagtacctctagctatgaaattgataca

SEQ ID NO: 60 - DNA, 16 bp, Artificial Sequence
  Primer
caatgcaaaagaaatt

SEQ ID NO: 61 - DNA, 25 bp, Artificial Sequence
  Primer
ggtccatttgctgctgtgtttaata

SEQ ID NO: 62 - DNA, 19 bp, Artificial Sequence
  Primer
atggaattagaggcaaaat

SEQ ID NO: 63 - DNA, 36 bp, Artificial Sequence
  Primer
aaacctaatgacatagataaacctccaaataaaagt

SEQ ID NO: 64 - DNA, 19 bp, Artificial Sequence
  Primer
atggaattagagacaaaat

SEQ ID NO: 65 - DNA, 40 bp, Artificial Sequence
  Primer
ttggttcatttgtattgttcataaaagtattatatttatg

SEQ ID NO: 66 - DNA, 21 bp, Artificial Sequence
  Primer
caaacttctttttgtattgtc

SEQ ID NO: 67 - DNA, 44 bp, Artificial Sequence
  Primer
atgatgaaaagaagaaattaaatgtatctgatagaaatatacct

SEQ ID NO: 68 - DNA, 21 bp, Artificial Sequence
  Primer
caaacttctttttttattgtc

SEQ ID NO: 69 - DNA, 36 bp, Artificial Sequence
  Primer
gtgaacataaatagtatcactatactgacttccatt

SEQ ID NO: 70 - DNA, 19 bp, Artificial Sequence
  Primer
ttggattcgacgtatgtat

SEQ ID NO: 71 - DNA, 39 bp, Artificial Sequence
  Primer
ggatgatgtttcattaaaaagcaaaaaattactatttcc

SEQ ID NO: 72 - DNA, 19 bp, Artificial Sequence
  Primer
ttggattcgacatatgtat

SEQ ID NO: 73 - DNA, 37 bp, Artificial Sequence
  Primer
cagatgaagaggataaagatgatgatgataatgataa

SEQ ID NO: 74 - DNA, 22 bp, Artificial Sequence
  Primer
catttctatcttttggttctat

SEQ ID NO: 75 - DNA, 40 bp, Artificial Sequence
  Primer
gaattatattatcctcatcattatgatttatattttcact

SEQ ID NO: 76 - DNA, 24 bp, Artificial Sequence
  Primer
ttcatttctatcttttagttctat

SEQ ID NO: 77 - DNA, 35 bp, Artificial Sequence
  Primer
gtttataattccattatttaaacgaacgacctcaa

SEQ ID NO: 78 - DNA, 21 bp, Artificial Sequence
  Primer
tgttctctttttttcgcttcg

SEQ ID NO: 79 - DNA, 40 bp, Artificial Sequence
  Primer
attattgtttatgcgtcattatatttttttacatgtgatt

SEQ ID NO: 80 - DNA, 22 bp, Artificial Sequence
  Primer
ttgttctcttttttttgcttcg

SEQ ID NO: 81 - DNA, 20 bp, Artificial Sequence
  Primer
ccagtttaccaagctttacg

SEQ ID NO: 82 - DNA, 21 bp, Artificial Sequence
  Primer
cagctattataaatggggatg

SEQ ID NO: 83 - DNA, 35 bp, Artificial Sequence
  Primer
tttgaaagtttaaaaattaaaacagcatttgatac

SEQ ID NO: 84 - DNA, 17 bp, Artificial Sequence
  Primer
tggctacaggcgtattt

SEQ ID NO: 85 - DNA, 38 bp, Artificial Sequence
  Primer
agaactaaacgaaatgacatacttatatattggaatgt

SEQ ID NO: 86 - DNA, 18 bp, Artificial Sequence
  Primer
atggctacaggtgtattt

SEQ ID NO: 87 - DNA, 41 bp, Artificial Sequence
  Primer
tcagagaatagtttaagtttatcaaaaaatagtgtttatgc

SEQ ID NO: 88 - DNA, 20 bp, Artificial Sequence
  Primer
ccaaaaacaaatgattatag

SEQ ID NO: 89 - DNA, 32 bp, Artificial Sequence
  Primer
acccccatccttctcatctatatattttacat

SEQ ID NO: 90 - DNA, 20 bp, Artificial Sequence
  Primer
ccaaaaacaaatggttatag

SEQ ID NO: 91 - DNA, 27 bp, Artificial Sequence
  Primer
tgtccttgctatatgaatacccagaga

SEQ ID NO: 92 - DNA, 23 bp, Artificial Sequence
  Primer
cctcattatatacatccaagtta

SEQ ID NO: 93 - DNA, 40 bp, Artificial Sequence
  Primer
gctgttaattatgacagaaaggatacaaaaaaaaataaat

SEQ ID NO: 94 - DNA, 23 bp, Artificial Sequence
  Primer
cctcattatatacattcaagtta

SEQ ID NO: 95 - DNA, 30 bp, Artificial Sequence
  Primer
tgtgtaagatttggtggatatctactgaga

SEQ ID NO: 96 - DNA, 18 bp, Artificial Sequence
  Primer
tggtatgtttgaagtttt

SEQ ID NO: 97 - DNA, 30 bp, Artificial Sequence
  Primer
aatggcaagaaagtcataatatctccgatt

SEQ ID NO: 98 - DNA, 19 bp, Artificial Sequence
  Primer
ttggtatgtttcaagtttt

SEQ ID NO: 99 - DNA, 35 bp, Artificial Sequence
  Primer
gcatcttgatacttctttgtattattattattatt

SEQ ID NO: 100 - DNA, 18 bp, Artificial Sequence
  Primer
caacttcctggttgtgtg

SEQ ID NO: 101 - DNA, 24 bp, Artificial Sequence
  Primer
gtgccttctcagaactcttgtctt

SEQ ID NO: 102 - DNA, 19 bp, Artificial Sequence
  Primer
tcaacttcctgattgtgtg

SEQ ID NO: 103 - DNA, 41 bp, Artificial Sequence
  Primer
acagaagagcaaatatataattcagaattaggtatatctga

SEQ ID NO: 104 - DNA, 18 bp, Artificial Sequence
  Primer
ttagtgaacagcaacatg

SEQ ID NO: 105 - DNA, 33 bp, Artificial Sequence
  Primer
tggcatctatttctattaagaaggttttttggt

SEQ ID NO: 106 - DNA, 17 bp, Artificial Sequence
  Primer
tagtgaacagaaacatg

SEQ ID NO: 107 - DNA, 20 bp, Artificial Sequence
  Primer
gacgttcagattgtgtttcc

SEQ ID NO: 108 - DNA, 20 bp, Artificial Sequence
  Primer
gacaaaaacttaacgcaagc

SEQ ID NO: 109 - DNA, 41 bp, Artificial Sequence
  Primer
gactctgattgatgggaagatttatattataattcatatca

SEQ ID NO: 110 - DNA, 21 bp, Artificial Sequence
  Primer
catttgtttgttgtacattat

SEQ ID NO: 111 - DNA, 36 bp, Artificial Sequence
  Primer
ccaagggaaaattttattcaataatgattctacaga

SEQ ID NO: 112 - DNA, 19 bp, Artificial Sequence
  Primer
tttgtttgttgtgcattat

SEQ ID NO: 113 - DNA, 20 bp, Artificial Sequence
  Primer
tcttttgaagcagaaacgat

SEQ ID NO: 114 - DNA, 20 bp, Artificial Sequence
  Primer
cgaattcaagagttgcactt

SEQ ID NO: 115 - DNA, 45 bp, Artificial Sequence
  Primer
ggaaccatataaaagtatatcaataaataatgtaaaaaggaaata

SEQ ID NO: 116 - DNA, 20 bp, Artificial Sequence
  Primer
attatgacaacatggtttaa

SEQ ID NO: 117 - DNA, 29 bp, Artificial Sequence
  Primer
ggagcattcaacgttaaagaattaccatt

SEQ ID NO: 118 - DNA, 16 bp, Artificial Sequence
  Primer
tgacaacacggtttaa

SEQ ID NO: 119 - DNA, 24 bp, Artificial Sequence
  Primer
gctgaaaccgaaattgaagaggat

SEQ ID NO: 120 - DNA, 18 bp, Artificial Sequence
  Primer
tctgatgatacgaataat

SEQ ID NO: 121 - DNA, 34 bp, Artificial Sequence
  Primer
ggtgatacactatttaattcatcagaatcttcca

SEQ ID NO: 122 - DNA, 20 bp, Artificial Sequence
  Primer
aatctgatgatactaataat

SEQ ID NO: 123 - DNA, 25 bp, Artificial Sequence
  Primer
gggatgagcgtatagatgaattggt

SEQ ID NO: 124 - DNA, 20 bp, Artificial Sequence
  Primer
atgaaactaacgaggtaatg

SEQ ID NO: 125 - DNA, 30 bp, Artificial Sequence
  Primer
tgtcctctacaaattcaacactgttaacat

SEQ ID NO: 126 - DNA, 20 bp, Artificial Sequence
  Primer
atgaaactaacgacgtaatg

SEQ ID NO: 127 - DNA, 36 bp, Artificial Sequence
  Primer
tctggaggcagatttatcaaaacatttatagattat

SEQ ID NO: 128 - DNA, 20 bp, Artificial Sequence
  Primer
atttgtacaaggatttaaat

SEQ ID NO: 129 - DNA, 29 bp, Artificial Sequence
  Primer
tctcgtcatttgaataaaagccacataga

SEQ ID NO: 130 - DNA, 18 bp, Artificial Sequence
  Primer
ttgtacaagggtttaaat

SEQ ID NO: 131 - DNA, 41 bp, Artificial Sequence
  Primer
tcacatagtatgaagatatatacaaatgaatggaacataac

SEQ ID NO: 132 - DNA, 22 bp, Artificial Sequence
  Primer
ccattatgtagaagtcaagata

SEQ ID NO: 133 - DNA, 35 bp, Artificial Sequence
  Primer
tttcccacattttttttacattccatttttataat

SEQ ID NO: 134 - DNA, 22 bp, Artificial Sequence
  Primer
ccattatgtagaagtaaagata

SEQ ID NO: 135 - DNA, 20 bp, Artificial Sequence
  Primer
gaatgttcagctggcgttat

SEQ ID NO: 136 - DNA, 21 bp, Artificial Sequence
  Primer
aacaaaaacatggactcgtga

SEQ ID NO: 137 - DNA, 25 bp, Artificial Sequence
  Primer
ttttatacaggtctacttcatttcg

SEQ ID NO: 138 - DNA, 21 bp, Artificial Sequence
  Primer
aaaggggaaatacacaaacat

SEQ ID NO: 139 - DNA, 20 bp, Artificial Sequence
  Primer
ccgtatgtgaagaagcacaa

SEQ ID NO: 140 - DNA, 20 bp, Artificial Sequence
  Primer
tcaggaggtaatgggtttga

SEQ ID NO: 141 - DNA, 20 bp, Artificial Sequence
  Primer
gtccccattcaggtttatca

SEQ ID NO: 142 - DNA, 24 bp, Artificial Sequence
  Primer
ttacagaggaaaaagaagaaaatg

SEQ ID NO: 143 - DNA, 24 bp, Artificial Sequence
  Primer
gcaaactacttagcagatacaacg

SEQ ID NO: 144 - DNA, 22 bp, Artificial Sequence
  Primer
tgaacgtcttctaatccttcct

SEQ ID NO: 145 - DNA, 24 bp, Artificial Sequence
  Primer
tgttgttaaaacagatgaagagga

SEQ ID NO: 146 - DNA, 20 bp, Artificial Sequence
  Primer
tgccatcactattttgttca

SEQ ID NO: 147 - DNA, 20 bp, Artificial Sequence
  Primer
ttttggaggtacaggggatt

SEQ ID NO: 148 - DNA, 24 bp, Artificial Sequence
  Primer
ttcagtgttctaaaatcaggtacg

SEQ ID NO: 149 - DNA, 24 bp, Artificial Sequence
  Primer
ccaagattacaacaaaatggatca

SEQ ID NO: 150 - DNA, 21 bp, Artificial Sequence
  Primer
gcgctttccgtaatatttttg

SEQ ID NO: 151 - DNA, 20 bp, Artificial Sequence
  Primer
gatagaaccagacgctccaa

SEQ ID NO: 152 - DNA, 23 bp, Artificial Sequence
  Primer
cattattatccccagaaatagga

SEQ ID NO: 153 - DNA, 20 bp, Artificial Sequence
  Primer
ccagcacacacttttgttga

SEQ ID NO: 154 - DNA, 20 bp, Artificial Sequence
  Primer
gaaacgccttatttggacag

SEQ ID NO: 155 - DNA, 23 bp, Artificial Sequence
  Primer
tcttttacaatatgttcccctga

SEQ ID NO: 156 - DNA, 20 bp, Artificial Sequence
  Primer
cccatgatgacatgaaacag

SEQ ID NO: 157 - DNA, 25 bp, Artificial Sequence
  Primer
tgcaaattatagaattcaaaaagag

SEQ ID NO: 158 - DNA, 21 bp, Artificial Sequence
  Primer
caataggtggctttaaaggat

SEQ ID NO: 159 - DNA, 20 bp, Artificial Sequence
  Primer
ggtagaagccaaatgtgacg

SEQ ID NO: 160 - DNA, 20 bp, Artificial Sequence
  Primer
caataggaggcatggaagaa

SEQ ID NO: 161 - DNA, 21 bp, Artificial Sequence
  Primer
tgaaaacaagacaatgcaaaa

SEQ ID NO: 162 - DNA, 22 bp, Artificial Sequence
  Primer
agacatgcatatgctgatcaat

SEQ ID NO: 163 - DNA, 20 bp, Artificial Sequence
  Primer
tattaacatcgggcgaagaa

SEQ ID NO: 164 - DNA, 23 bp, Artificial Sequence
  Primer
cgttaaagtgttcatcatcatcc

SEQ ID NO: 165 - DNA, 21 bp, Artificial Sequence
  Primer
ttaatacgtgggttcgtttca

SEQ ID NO: 166 - DNA, 21 bp, Artificial Sequence
  Primer
ccacaaaagaaatatcgagca

SEQ ID NO: 167 - DNA, 22 bp, Artificial Sequence
  Primer
tcatcatatggtaacatggaca

SEQ ID NO: 168 - DNA, 22 bp, Artificial Sequence
  Primer
tcgataatacaaagagcgttca

SEQ ID NO: 169 - DNA, 20 bp, Artificial Sequence
  Primer
aatggcaagaagttgaatcg

SEQ ID NO: 170 - DNA, 22 bp, Artificial Sequence
  Primer
gcacaggcaactaatacaaatg

SEQ ID NO: 171 - DNA, 26 bp, Artificial Sequence
  Primer
ttccatgttattaatatatcagcatt

SEQ ID NO: 172 - DNA, 26 bp, Artificial Sequence
  Primer
gctaaggaaaatggaagtatagaaaa

SEQ ID NO: 173 - DNA, 19 bp, Artificial Sequence
  Primer
aaatcgggggatgaagaag

SEQ ID NO: 174 - DNA, 20 bp, Artificial Sequence
  Primer
ttcgtccaatggtttctgat

SEQ ID NO: 175 - DNA, 24 bp, Artificial Sequence
  Primer
ttgtgaaatattgaaatatgaagc

SEQ ID NO: 176 - DNA, 22 bp, Artificial Sequence
  Primer
aggatcacaaatccataactgt

SEQ ID NO: 177 - DNA, 20 bp, Artificial Sequence
  Primer
tatgccatagcgttatccaa

SEQ ID NO: 178 - DNA, 20 bp, Artificial Sequence
  Primer
ttttgttgtcccatttttga

SEQ ID NO: 179 - DNA, 20 bp, Artificial Sequence
  Primer
tgttctttttccttgtgtcg

SEQ ID NO: 180 - DNA, 22 bp, Artificial Sequence
  Primer
tggagttaaataatccctttgt

SEQ ID NO: 181 - DNA, 21 bp, Artificial Sequence
  Primer
tcagaagaacttgaaccatcc

SEQ ID NO: 182 - DNA, 20 bp, Artificial Sequence
  Primer
catctccttcagcatcttca

SEQ ID NO: 183 - DNA, 20 bp, Artificial Sequence
  Primer
gcaggcattaaatgtccata

SEQ ID NO: 184 - DNA, 20 bp, Artificial Sequence
  Primer
cttttgcgaatcgatttctt

SEQ ID NO: 185 - DNA, 23 bp, Artificial Sequence
  Primer
aacaattcacataccacttaccc

SEQ ID NO: 186 - DNA, 20 bp, Artificial Sequence
  Primer
tccgcatatctatcatttcg

SEQ ID NO: 187 - DNA, 20 bp, Artificial Sequence
  Primer
ggtgtgtggaaacaggaaat

SEQ ID NO: 188 - DNA, 20 bp, Artificial Sequence
  Primer
ccattatcatacccagcaca

SEQ ID NO: 189 - DNA, 21 bp, Artificial Sequence
  Primer
ttgtgatagagaaggggaaca

SEQ ID NO: 190 - DNA, 22 bp, Artificial Sequence
  Primer
cagctgaaaactgagctctatg

SEQ ID NO: 191 - DNA, 20 bp, Artificial Sequence
  Primer
gggtggttatatgtcagcag

SEQ ID NO: 192 - DNA, 20 bp, Artificial Sequence
  Primer
atgtttttcgggagatacga

SEQ ID NO: 193 - DNA, 19 bp, Artificial Sequence
  Primer
ccgaaacgcccttataaat

SEQ ID NO: 194 - DNA, 21 bp, Artificial Sequence
  Primer
aatgccaaatcaaaactgtct

SEQ ID NO: 195 - DNA, 20 bp, Artificial Sequence
  Primer
tggcaagcgaaaaatataaa

SEQ ID NO: 196 - DNA, 20 bp, Artificial Sequence
  Primer
caacctcttcttcctgcttc

SEQ ID NO: 197 - DNA, 20 bp, Artificial Sequence
  Primer
atgaattccccaattcaaag

SEQ ID NO: 198 - DNA, 20 bp, Artificial Sequence
  Primer
atcgtgttcctccagctaat

SEQ ID NO: 199 - DNA, 21 bp, Artificial Sequence
  Primer
tttcctcaatattacgggtga

SEQ ID NO: 200 - DNA, 20 bp, Artificial Sequence
  Primer
aattctttggcattcatgtg

SEQ ID NO: 201 - DNA, 20 bp, Artificial Sequence
  Primer
ataaattcggatgaggatgc

SEQ ID NO: 202 - DNA, 20 bp, Artificial Sequence
  Primer
catcgacccatttacaacct

SEQ ID NO: 203 - DNA, 20 bp, Artificial Sequence
  Primer
gaacaacccaatcttgttga

SEQ ID NO: 204 - DNA, 21 bp, Artificial Sequence
  Primer
tctctcatccgttttaattgg

SEQ ID NO: 205 - DNA, 20 bp, Artificial Sequence
  Primer
tgtgagtgagaatggacctg

SEQ ID NO: 206 - DNA, 20 bp, Artificial Sequence
  Primer
tttttcaaagttggacgtgt

SEQ ID NO: 207 - DNA, 20 bp, Artificial Sequence
  Primer
attagaatacggtgccctga

SEQ ID NO: 208 - DNA, 22 bp, Artificial Sequence
  Primer
tgcatagcgaagtatcatatcc

SEQ ID NO: 209 - DNA, 21 bp, Artificial Sequence
  Primer
ttgcagttgactgttgtagga

SEQ ID NO: 210 - DNA, 20 bp, Artificial Sequence
  Primer
tggtaagggggtatgtgaat

SEQ ID NO: 211 - DNA, 22 bp, Artificial Sequence
  Primer
caaatgaaactacccctaatga

SEQ ID NO: 212 - DNA, 20 bp, Artificial Sequence
  Primer
ttgaaaccccatttgttttt

SEQ ID NO: 213 - DNA, 20 bp, Artificial Sequence
  Primer
atggtcccatagcacaagag

SEQ ID NO: 214 - DNA, 20 bp, Artificial Sequence
  Primer
tcgaacatttcatctttgga

SEQ ID NO: 215 - DNA, 20 bp, Artificial Sequence
  Primer
cgaaaaacagcagctcaata

SEQ ID NO: 216 - DNA, 20 bp, Artificial Sequence
  Primer
ccttttcatcgcaggtagtt

SEQ ID NO: 217 - DNA, 20 bp, Artificial Sequence
  Primer
gatgctgctggaaaaactac

SEQ ID NO: 218 - DNA, 20 bp, Artificial Sequence
  Primer
cctacatcccatacggtaaa

SEQ ID NO: 219 - DNA, 20 bp, Artificial Sequence
  Primer
tcatgcatcgctactggaag

SEQ ID NO: 220 - DNA, 19 bp, Artificial Sequence
  Primer
ccagaaaagtccaccttgg

SEQ ID NO: 221 - DNA, 20 bp, Artificial Sequence
  Primer
ccgcttctacaacacgatga

SEQ ID NO: 222 - DNA, 19 bp, Artificial Sequence
  Primer
cgaccagatgggcaagtat

SEQ ID NO: 223 - DNA, 20 bp, Artificial Sequence
  Primer
agaaccagcagaccaccatc

SEQ ID NO: 224 - DNA, 20 bp, Artificial Sequence
  Primer
gctgcaaacttcggctattc

SEQ ID NO: 225 - DNA, 18 bp, Artificial Sequence
  Primer
gtttctcaccgcgttcgt

SEQ ID NO: 226 - DNA, 20 bp, Artificial Sequence
  Primer
aaccaatccaatgccttctc

SEQ ID NO: 227 - DNA, 44 bp, Artificial Sequence
  Primer
agctggagctccaccgcggtttataaaaacattcctaacacatt

SEQ ID NO: 228 - DNA, 38 bp, Artificial Sequence
  Primer
cgggggatccactagtatttaccttacatttatctcca

SEQ ID NO: 229 - DNA, 23 bp, Artificial Sequence
  Primer
agtatgcaataaattcatcgttc

SEQ ID NO: 230 - DNA, 26 bp, Artificial Sequence
  Primer
atataagaacatatttattaaactgc

(19) United States
(12) Patent Application Publication
(10) Pub. No.: US 2015/0203547 A1
(43) Pub. Date: Jul. 23, 2015

(54) USE OF P47 FROM PLASMODIUM FALCIPARUM (PFS47) OR PLASMODIUM VIVAX (PVS47) AS A VACCINE OR DRUG SCREENING TARGETS FOR THE INHIBITION OF HUMAN MALARIA TRANSMISSION

(71) Applicant: The United States of America, as represented by the Secretary, Dept. of Health and Human Services, Bethesda, MD (US)

(72) Inventors: Carolina Veronica Barillas-Mury, Silver Spring, MD (US); Alvaro Molina-Cruz, Bethesda, MD (US)

(73) Assignee: The United States of America, as represented by the Secretary, DHHS, Bethesda, MD (US)

(21) Appl. No.: 14/421,964

(22) PCT Filed: Aug. 16, 2013
(86) PCT No.: PCT/US2013/055372

§ 371 Date: Feb. 16, 2015

Related U.S. Application Data
(60) Provisional application No. 61/684,333, filed on Aug. 17, 2012.

(51) Int. Cl. C07K 14/445; C07K 16/20; A61K 39/015

(52) U.S. Cl. CPC C07K 14/445; A61K 39/015; C07K 16/205; A61K 38/00

(57) ABSTRACT

The inventors have identified Pfs47, a gene from the malaria parasite P. falciparum, as a key factor for survival of these parasites in the mosquito Anopheles gambiae. A. gambiae is a major natural vector of human malaria in Africa. The Pfs47 protein may allow the parasite to survive in the mosquito by manipulating the mosquito's immune system. The inventors propose the use of P47 proteins, including Pfs47 and Pvs47 as a target of vaccines or pharmaceutical agents that will block or reduce P. falciparum or P. vivax infection in A. gambiae or other anopheline mosquitoes and thus prevent further transmission of the parasites in humans.

### CROSS-REFERENCE TO RELATED APPLICATIONS

This application claims the priority benefit of U.S. Provisional Application No. 61/684,333, filed Aug. 17, 2012, all of which are incorporated herein by reference in their entirety.

### FIELD OF THE INVENTION FIELD OF THE INVENTION

The present invention comprises methods and compositions for delivering a Plasmodium P47 vaccine, or an antibody to P47 to prevent Plasmodium falciparum or Plasmodium vivax malaria. The P47 vaccine, antibody vaccine or drug that blocks or reduces transmission of the parasite in anopheline mosquitoes, thereby renders ineffective the vector most responsible for the malaria epidemic.

### BACKGROUND OF THE INVENTION

Human malaria is caused by Plasmodium parasites. According to the Centers for Disease Control, malaria is one of the most severe public health problems worldwide. It is the leading cause of death and disease in many developing countries, affecting mostly young children and pregnant women. Over three billion people (half of the world's population) live in areas at risk of malaria transmission in 109 countries and territories. Thirty-five countries (30 in sub-Saharan Africa and 5 in Asia) account for 98% of global malaria deaths. The WHO estimates that in 2008, malaria caused 190-311 million clinical episodes and 708,000-1,003,000 deaths. Eighty-nine percent of the malaria deaths worldwide occur in Africa. Malaria is the fifth most prevalent cause of death from infectious diseases worldwide (after respiratory infections, HIV/AIDS, diarrheal diseases, and tuberculosis). Malaria is the second leading cause of death from infectious diseases in Africa, after HIV 1 AIDS.

The mosquito Anopheles gambiae is the main vector of Plasmodium falciparum malaria in large areas of sub-Saharan Africa. Mosquitoes become infected when they ingest blood from an infected human, and the parasites need to undergo a complex developmental cycle in the mosquito to be transmitted to another person. Work from animal models indicates that mosquitoes can defend themselves by mounting immune responses that can greatly limit Plasmodium survival. Thus, it is not clear why disease transmission is so effective in highly endemic areas. In all regions of the world where malaria has been eliminated, this has been achieved by controlling the mosquito vector populations, and reducing the rate of disease transmission is considered one of the key steps to eliminate malaria from endemic areas.

Currently, there is no FDA-approved vaccine available for malaria, and there is growing resistance to existing anti-malarial drugs. Current therapies for malaria infection include chloroquine, quinine sulfate, hydroxychloroquine, mefloquine, doxycycline, and artemisinin. Chloroquine resistance is well-known; resistance to artemisinin has been reported. A transmission-blocking vaccine will reduced disease burden, thus increasing the effectiveness of current therapies.

Research has been and is being conducted in the area of P. falciparum gamete surface proteins, often with the objective of identification of a malaria vaccine candidate. During its life cycle, P. falciparum alternates between mosquito and human hosts. When an infected mosquito takes a blood meal, the parasite sporozoite infects liver of the human host, multiplies and takes up residence in red blood cells (RBCs), wherein they continue to multiply and some of them develop into gametocytes. When the RBCs are taken up by the mosquito with the next blood meal, the gametocytes exit the RBCs as gametes in the mosquito midgut. Fertilization ensues to form a zygote which, in due course, develops into a motile ookinete. Ookinetes transform in to oocysts, a stage in which the parasite multiplies and releases thousands of sporozoites that migrate to the mosquito salivary gland and will infect a new person when the infected mosquito takes a blood meal.

Sexual stage-specific surface antigens are of interest as vaccine candidates, because disruption of these antigens would reduce the fertility and, hence, the infectivity of the parasite. In Eksi, S. et al. (2006) Molec. Microbiol. 61(4):991-998, it was found that Pfs230 antigen, found on the surface of male gametes, has the sexual-stage paralogs Pfs48/45 and Pfs47. Pfs230 is noted to be an important vaccine candidate due to its role in RBC interaction and oocyst production. Pfs47 was assayed as a structural analog to Pfs48/45 for reactivity with surface of intact Pfs230Δ1 macrogametes. In Gerloff, D L et al. (2005) Proc. Natl. Acad. Sci. 102(38):13598-13603, Pfs230 is identified as the largest protein of a Plasmodium 10-member family characterized by cysteine-rich double domains with 1-3 di-S bridges in each half. Pfs230, Pfs 48/45, Pfs47 and others are mentioned as potential transmission-blocking vaccines due to their surface location on gametes.

In Anthony, T G et al. (2007) Mol. Biochem. Parasitol. 156(2):117-23, sequence non-synonymous polymorphism was observed for Pfs47 and Pfs48/45 and was suggested to be functionally important to fertility. In van Schaijk, B C L et al. (2006) Molec. Biochem. Parasitology 149(2):216-222, the importance of Pfs48/45 and Pfs230 as candidate vaccines is discussed due to their localization on gamete surfaces. Paralog Pfs47, though expressed on female gametes, appears, from knock-out and monoclonal antibody experiments, not to be important to fertility. Specifically, three monoclonal antibodies against Pfs47 were unable to inhibit Anopheles stephens mosquito infection when they were added to blood meals containing wild type parasites. The authors conclude that Pfs47 is not a likely vaccine candidate.

Research has also been conducted to define the mechanism by which some Plasmodium parasites evade the mosquito immune system. It has been reported that mosquitoes refractory to infection by some lines of Plasmodium falciparum can melanize other lines. Specifically, Anopheles gambiae L3-5 refractory line melanizes the Brazilian Plasmodium falciparum 7G8 line, but not the African Plasmodium falciparum 3D7, NF54 and GB4 strains. Investigation of this difference in parasite ability to infect the same refractory line suggested a role for Thioester containing protein 1 (TEP1) in the mosquito's capacity to inhibit Plasmodium transmission (Molina-Cruz (2012) PNAS 109(28): E1957-E1962).

### SUMMARY OF THE INVENTION

Plasmodium falciparum has several strains that have been isolated including those found in Africa, America and Asia. Each of these strains expresses a slightly different version of the Pfs47 protein. The subject invention comprises the recognition that Pfs47 allows the parasite to suppress or evade the immune system, thereby ensuring the parasite's survival. The evolution of Pfs47 provides a very powerful mechanism by which the effective transmittal of Plasmodium falciparum is permitted in the field.

While the gene encoding Pfs47 is known, the fact that Pfs47 enables survival of the parasite by manipulation of the mosquito immune system, has not been previously understood. As this relationship has now been discovered, it has become possible to develop new malaria vaccines and methods for identifying additional pharmaceutical agents that can interfere with the capacity of Pfs47 to manipulate the mosquito immune system.

Reducing the rate of malaria transmission is essential to eliminate the disease. As mentioned herein, there has been reduced support for Pfs47 as a transmission-blocking target. Antibodies to Pfs47 were unable to inhibit mosquito infection when they were added to blood meals for wild type parasites (Schaijk et al. (2006), supra). In contrast to the accepted view in the prior art, the subject invention comprises the recognition that Pfs47 is, in fact, critical to the transmission of P. falciparum in the field. The novel use of Pfs47 and related compounds as transmission-blocking targets involves active participation of the mosquito immune system. Specifically, contact of a vaccine comprising Pfs47 or an antibody thereto, or a pharmaceutical agent that inhibits Pfs47, with the mosquito can prevent Pfs47 from interacting with and manipulating the mosquito immune system. By inhibiting or inactivating Pfs47 via such vaccine or agent, the mosquito immune system is able to “see” the parasite and destroy or substantially reduce it.

The subject invention further comprises the recognition that P47 proteins, their antibodies and pharmaceutical agents found to be inhibitory to P47, can be effective as vaccines or transmission-blocking agents of malaria transmission. As is discussed herein, P47 proteins include, without limitation, Pfs47 produced by Plasmodium falciparum, and Pvs47 produced by Plasmodium vivax.

As is detailed in the Examples, a refractory strain of A. gambiae that cannot kill P. falciparum African strains GB4, NF54 and 3D7 but is very effective killing the Plasmodium falciparum Brazilian 7G8 strain, was used to identify Pfs47 as the protein responsible for efficient transmission of P. falciparum. Based on the critical role of Pfs47 in transmission, it became evident that disruption of the function of Pfs47 by various means can be an innovative and forceful means to substantially control and reduce the malaria epidemic.

Thus, as is detailed hereinbelow, one embodiment of the subject invention comprises vaccines and their administration, wherein the vaccines are pharmaceutical compositions comprising P47 protein (or immunological fragments or variants thereof); or pharmaceutical compositions comprising antibodies (or fragments thereof) to P47 protein (or its immunological fragments or variants).

In other embodiments, the critical role of P47 is exploited to develop new pharmaceutical agents that can be used to disrupt P47 function in the field. In one aspect, the assay for identification of new agents comprises the screening of candidate compounds in Plasmodium falciparum infected mosquitoes. In another aspect, the assay involves screening of candidate compounds in a cell in which the JNK signaling pathway has been inactivated to identify those agents that can restore JNK signaling.

In further embodiments, the invention includes transgenic and paratransgenic mosquitoes capable of expressing antibodies or other pharmaceutical agents that can disrupt P47 function. In paratransgenic mosquitoes, the gene encoding the antibody or other pharmaceutical agent has been inserted into the genome of bacteria resident in the gut microbiota and expressed and exported, whereby the agent can interact with the P47 in the mosquito gut or other organs.

### BRIEF DESCRIPTION OF THE DRAWINGS

FIG. 1 shows Survival of the parental and progeny Plasmodium falciparum lines in refractory (R) mosquitoes and quantitative trait locus (QTL) mapping of the melanization phenotype. (A) P. falciparum GB4 and 7G8 parasites in the midgut of R mosquitoes. (B) Melanization phenotype of the parental and progeny lines of the GB4×7G8 genetic cross in R mosquitoes. (C) Logarithm of odds scores (LOD) of genome-wide QTL analysis of the melanization phenotype. Red dotted lines, statistical significance thresholds at P=0.05 and P=0.40; arrow, significant QTL.

FIG. 2 shows linkage group selection, mRNA expression, and coding region sequence analysis. (A) Genotype frequency of homozygous African (AA; horizontal line), Brazilian (BB; vertical line), or heterozygous (AB, diagonal dashed line) markers along Chr. 13 in individual oocysts dissected from S or R mosquitoes. (black arrow, region with BB under extreme negative selection in R strain). (B) Relative mRNA expression of candidate genes in GB4/7G8 Plasmodium falciparum ookinete stage. Magenta dots, genes with non-synonymous single nucleotide polymorphisms (SNPs) between GB4 and 7G8; Arrows, SNPs shared between GB4-3D7 and 7G8-SL strains.

FIG. 3 shows phenotype of NF54 wild type (WT) or Pfs47 knockout (KO) on different mosquitoes. (A) Number of melanized (x-axis) and live (y-axis) KO parasites in R and S mosquitoes. Each dot represents an individual midgut. Medians are indicated by the horizontal lines. (B) Effect of TEP-1 silencing on KO infection in R mosquitoes. (C) Effect of silencing TEP1 on WT and KO infection in S mosquitoes. (D) Immunofluorescence staining of WT and KO ookinetes with Pfs47 and Pfs25 (scale bar=5 μm). (E) Midgut mRNA expression of HPX2 and NOX5, and midgut nitration using ELISA, 24 h after S females were infected with WT or KO parasites (I=infected) or fed uninfected blood (C=control).

FIG. 4 shows Effect of complementing Pfs47 knockout (KO) parasites with the Brazilian (7G8) and African (NF54) alleles of Pfs47. Infectivity of Pfs47 KO parasites complemented with the (A) NF54 or (B) 7G8 Pfs47 alleles in the Anopheles gambiae R strain.

FIG. 5 shows Graphic representation of the markers that defined the chromosome 13 quantitative trait locus (QTL) associated with the Plasmodium falciparum (Pf) melanization phenotype. Microsatellite (MS) marker genotype of Pf parental (GB4 allele, diagonal line; 7G8 allele, diagonal dashed line) and three progeny lines (KA6, DAN, and WE2) that have recombination sites near the initial QTL boundaries. Markers already known based on the published linkage map for this cross are indicated by their name (C13M63, C13M87, and TA56) (1). New MS markers were identified from the genome and used to map the recombination sites more precisely; their chromosomal location is indicated in Kb (top row). The size of the PCR product for the allele of each marker is indicated in base pairs. Final QTL boundaries identified by the recombination sites (1773.4 Kb and 1945.68 Kb) are indicated by arrows (↓) and define a 172-Kb region. The sequences of the primers used for genotyping are shown in Table 1.

FIG. 6 shows genotype along chromosome 13 of individual oocysts dissected from the midgut of susceptible (S) G3 or refractory (R) L3-5 Anopheles gambiae mosquitoes infected with the un-cloned progeny of the cross between the GB4 African (A allele) and the 7G8 Brazilian (B allele) Plasmodium falciparum strains. The genotype of individual oocysts for each marker is indicated by different shading patterns: homozygous African (AA, in diagonal line), Brazilian (BB, in diagonal dashed line), and heterozygous (AB, in grid pattern). Frequency of genotypes for each marker is indicated at the bottom of each table. The chromosomal location and primers for the 26 markers along chromosome 13 are shown in Table 2. The QTL boundaries are indicated by the arrows.

FIG. 7 shows genotype of 50 additional individual oocysts dissected from refractory L3-5 Anopheles gambiae mosquitoes infected with the un-cloned progeny of the cross between the GB4 African and 7G8 Brazilian Plasmodium falciparum strains. Genotyping was done in the region of chromosome 13 under strong genetic selection. The genotype of individual oocysts for each marker is indicated by different shading patterns: homozygous African (AA, in diagonal line), Brazilian (BB, in grid pattern), and heterozygous (AB, in diagonal dashed line). The chromosomal location and primers for the markers along chromosome 13 are shown in Table 2. The QTL boundaries are indicated by the arrows.

FIG. 8 shows phenotype of Pfs48/45 knockout (KO) NF54 Plasmodium falciparum parasites in the An. gambiae refractory (R) strain. Number of melanized (x-axis) and live (y-axis) Pfs48/45 KO parasites per midgut in R mosquitoes. Medians are indicated by black lines (—).

FIG. 9 shows phenotype of Pfs47 knockout (KO) NF54 Plasmodium falciparum parasites in Anopheles stephensi (Nijmegen Sda500 strain) mosquitoes. Number of melanized (x-axis) and live (y-axis) Pfs47 KO parasites per midgut in refractory mosquitoes. Medians are indicated by black lines (—). This infection was done with the same gametocyte culture as that shown in FIG. 3A (left panel) with Anopheles gambiae susceptible strain mosquitoes. The intensity of infection in An. stephensi (Nijmegen) was significantly higher (median of 60 oocysts/midgut) than that in An. gambiae susceptible females (median of 1 oocyst/midgut; P<0.0001).

FIG. 10 illustrates the pCBM-BSD plasmid with the Pfs47 gene used for complementation of Pfs47 KO parasites. The short arrows indicate the primers used to test the presence of the plasmid by PCR in the Pfs47 KO complemented (BSD 3′ and 0248_b_F). Pfs47 sequence including ORF and contiguous regions are indicated in thick diagonal line and thin diagonal line respectively.

FIG. 11 shows PCR-based confirmation of the Pfs47 KO genetic background and the presence of the pCBM-BSD plasmid in the complemented Pfs47 KO lines DNA. The PCR products using primers BVS01 and L430 confirmed the Pfs47 KO background and the PCR products with primers BSD 3′ and 0248_b_F confirmed the presence of the pCBM-BSD plasmid containing the corresponding Pfs47 alelles, 7G8 or NF54 (3D7 clone). The PCR reactions using NF54 (Wt) and Pfs47 KO lines genomic DNA templates and the no-template-control (NTC) were included as controls.

FIG. 12 shows confirmation of the Pfs47 KO background and the Pfs47 mRNA expression upon genetic complementation. Relative mRNA expression of Pfs47 in wild-type (NF54), Pfs47 knockout (KO), and the KO line complemented with the NF54 or 7G8 allele of Pfs47. Relative mRNA expression of Pfs47 was assessed by qPCR in stage IV-V gametocyte cultures. Detection of Pfs47 mRNA in the complemented lines confirms gene expression upon complementation of the Pfs47 KO line.

FIG. 13 shows confirmation of the Pfs47 KO background and the Pfs47 protein expression upon genetic complementation. Western blot analysis of expression of Pfs47 protein in equivalent amounts of gametocyte cultures of wild-type (NF54), Pfs47 KO, and Pfs47 KO line complemented with the NF54 or 7G8 allele. Detection of Pfs47 protein in the complemented lines confirms gene expression upon complementation of the Pfs47 KO line.

FIG. 14 shows effect of removing the complementation selection (blasticidin) in Pfs47 knockout (KO) parasites complemented with the African (NF54) alleles of Pfs47. Parasites (live and dead) per midgut in A. gambiae R strain infected with gametocytes of Pfs47 KO complemented with the NF54 Pfs47. The complementation selection drug (blasticidin) was removed 1 week before setting up the gametocyte culture, giving raise to melanotic phenotype together with the complemented live parasites. Medians are indicated by black lines (—).

### DETAILED DESCRIPTION OF THE INVENTION

In the description that follows, a number of terms used in parasitology, immunology, and recombinant DNA technology are used. In order to provide a clear understanding of the terms, the following definitions are provided.

“P47” protein refers generically to those proteins produced by the Plasmodium genus of parasites that can or may enable the parasites to manipulate, inhibit or other avoid the immunological repression by mosquitoes. It includes, without limitation, Pfs47 (SEQ ID NO:1), produced by Anopheles gambiae, and Pvs47 (SEQ ID NO:2), produced by Plasmodium vivax.

SEQ ID NO: 1
Pfs47 Plasmodium falciparum 3D7 strain 439 aa
ACCESSION No: XP_001350182
MCMGRMISIINIILFYFFLWVKKSISELLSSTQYVCDFYFNPLTNVKPTV
VGSSEIYEEVGCTINNPTLGDHIVLICPKKNNGDFSNIEIVPTNCFESHL
YSAYKNDSSAYHLEKLDIDKKYAINSSFSDFYLKILVIPNEYKSHKTIYC
RCDNSKTEKNIPGQDKILKGKLGLVKIILRNQYNNIIELEKTKPIIHNKK
DTYKYDIKLKESDILMFYMKEETIVESGNCEEILNTKINLLSNNNVVIKM
PSIFINNINCMLSSQDQNNEKNYINLKADKTKHIDGCDFTKPKGKGIYKN
GFIINDIPNEEERICTVHLWNKKNQTIAGIKCPYKLIPPYCFKHVLYEKE
IDSQKTYKTFLLSDVLDTPNIEYYGNNKEGMYMLALPTKPEKTNKIRCIC
EQGGKKAVMELHIASTSTKYISMFLIFFLIVIFYMYVSI
SEQ ID NO: 2
Pvs47 Plasmodium vivax Sal-1 strain 433 aa
ACCESSION No: XP_001614247
MKLLTFAAATYGFLLKECLNSFIFPTKHLCDFALNPHSSIKPVLKEASGK
DEEVWCSVHNPSLTDYVAMVCPKKKGGDYTELETVPANCFTKHLYSPYDS
EENEKDMELLELDPKLSFNRTFNDFVLKVLVIPGYYKHNKTIYCRCDNRK
TKKGEDQEKIEEGKVGLVKIVLNKKEKKPRGIDFTETDELEQTDIVQNGN
DKLVKVKENETIHFKFNSNQKLEIKECENVINMKYGFLQEHVLNFRFPAV
FLSSENCTITVIESAKTPVRIIIKTQKTENIDGCDFTKPSGEGDYQDGFA
LEELKSNEKICTIHIGSSKKKISAGIKCPYKLTPTYCFRHVLYEKDVNGV
KSYHPFLLTDVLGTLDVEFYSNAQEGSYIIGLPTNPQKYSVVRCVCEHNG
KAGIMELRIASSSGWAFLSLTLLLLLIALLSAC

The “Plasmodium” genus of parasites include, without limitation, Plasmodium falciparum (P. falciparum), Plasmodium vivax (P. vivax), Plasmodium knowlesi (P. knowlesi), Plasmodium ovale (P. ovale), Plasmodium malariae (P. malariae), Plasmodium berghei (P. berghei), Plasmodium chabaudi (P. chabaudi), Plasmodium gallinaceum (P. gallinaceum), Plasmodium reichenowi (P. reichenowi) and Plasmodium yoelii (P. yoelii). The species most common for human malaria transmission are P. falciparum, P. vivax, P. knowles, P. ovale and P. malariae.

“Mosquitoes” that are commonly susceptible or vulnerable to infection by the Plasmodium parasites, include the Anopheles genus, which includes, without limitation, the species Anopheles gambiae (A. gambiae), Anopheles albimanus (A. albimanus), Anopheles darling (A. darlingi), Anopheles aquasalis (A. aquasalis), Anopheles freeborni (A. freeborni), Anopheles quadrimaculatus (A. quadrimaculatus) and Anopheles stephensi (A. stephensi).

An “epitope” is generally defined as a linear array of 3-10 amino acids aligned along the surface of a protein. A conformational epitope has residues that are not joined sequentially, but lie linearly along the surface due to the conformation (folding) of the protein. In either case, the epitope is immunoreactive.

“Immunoreactive,” as used herein, means that the epitope or antigen in question will react specifically with antibodies of interest and, preferably, anti-P47 antibodies present, for example, in a biological sample from an individual having malaria.

“Immunogenic,” as used herein, means the ability of a substance to cause a cellular and/or humoral response. More specifically, immunogenic refers to the ability of a polypeptide to generate antibody that blocks malaria transmission. The substance may be linked to a carrier and may be admixed with an adjuvant.

A “vaccine,” as used herein, means an immunogenic composition capable of eliciting partial or complete protection against malaria. A vaccine can be prophylactic for infection and/or therapeutic in an infected individual.

A “variant” of an original polypeptide is one which has at least about 80% identity to the sequence of the original polypeptide or an immunogenic fragment of the original polypeptide, and which substantially retains the desired effect on the intended target of the original polypeptide (i.e., elicits an immunogenic response). In increasingly preferred embodiments, the variant has at least about 85%, 90%, 95%, 97% or 99% identity to the sequence of the original polypeptide or an immunogenic fragment thereof.

“Antibody” means a protein or immunoglobulin (Ig) produced by B cells of the humoral immune system in the body in response to the presence of an antigen. An antibody can also refer to polyclonal and monoclonal antibodies or to any active form of the antibody, including Fab and F(ab′)₂ fragments and chimeric antibodies. Monoclonal antibodies can be obtained by methods known in the art (Kohler & Milstein (1975) Nature 256:495-497). Methods for production of antibodies in a variety of expression systems (plants, animals, and insects) are known in the art. Where an antibody is to be administered to a recipient species, it is preferred that they be compatible, so that the antibodies are not cleared before the parasite can be controlled. It is also preferred that the administered antibodies do not cause “serum sickness” in the individual.

A “fragment” of an antibody refers to an antibody polypeptide fragment, e.g., Fab and F(ab′)₂ fragments, capable of binding to the intended target and executing the desired effect, e.g., inhibition of Pfs47 function.

“Biological sample” means a fluid or tissue of an individual that commonly contains antibodies produced by the individual, more particularly antibodies against malaria. The tissue or fluid can also contain P. falciparum antigen. Biological samples include, without limitation, blood, plasma, serum, white blood cells, myelomas, tears, saliva, milk, urine, spinal fluid, lymph fluid, respiratory secretions, and genitourinary or intestinal tract secretions.

As used herein, “substantially reducing” or “substantially blocking” are interchangeable, and can be used in reference to destruction, killing, or reduction in transmission of the Plasmodium parasite, e.g., P. falciparum or P. vivax, in mosquitoes, such as A. gambiae. Preferably, the reduction in transmission is at least 10%, and, with increasing degrees of preference, is at least 20%, 30%, 40%, 50%, 60%, 70%, 80%, 90%, and is most preferably 100%. Reduction in transmission of the parasite in mosquitoes can be determined by methods known in the art including without limitation, the Standard Membrane Feeding Assay (SMFA). The SMFA is a functional assay that measures the ability of antibodies to block transmission of parasites to mosquitoes (see www.malariavaccine.org/files/MVIfactsheets_gia.pdf; and van der Kolk M et al. (2005) Parasitology 130(Pt. 1):13-22.)

“Solid phase” refers to a solid body to which the P. falciparum antigen or other compound or complex of interest is bound by covalent or non-covalent means such as by van der Waals, hydrophobic or ionic interaction.

The term “purified,” as used herein, with respect to polypeptides (proteins) or polynucleotides means a composition in which the molecule of interest is, with increasing preference, at least 40% of the total matter in the composition, at least 50% of the total matter, at least 60% of the total matter, at least 70% of the total matter, at least 80% of the total matter, at least 90% of the total matter, or at least 95% of the total matter.

An “essentially purified” polypeptide (protein) or polynucleotide is a polypeptide or polynucleotide that is substantially free from cellular matter that is not of interest and has been purified to homogeneity. With increasing preference, an essentially purified molecule is at least 80% pure, at least 90% pure, at least 95% pure, at least 97% pure, at least 98% pure, at least 99% pure, or most preferably is 100% pure.

A “polypeptide,” as used herein, means a polymer of amino acids of unspecified length and can include proteins. It can include modified and unmodified polypeptides.

A “recombinant polynucleotide or nucleic acid” refers to a polynucleotide or nucleic acid that is the result of splicing of two or more different sources, such as the splicing of genes from different organisms or of a gene with non-natural nucleic acid.

A “vector,” as used in the context of cellular transfection or transformation, is an autonomous polynucleotide replication unit within a cell that comprises sequences for expression of a desired polynucleotide.

The term “effective amount” for prophylactic or therapeutic treatment refers to an amount of epitope-bearing polypeptide (e.g., of Pfs47) sufficient to elicit an immunogenic response in the individual or an amount of antibody fragment sufficient to bind to and execute the desired effect on the intended target (e.g., Pfs47). It is believed that the effective amount(s) can be found within a relatively large, non-critical range. Routine experimentation can be used to determine appropriate effective amounts.

A “pharmaceutically acceptable carrier” is a carrier of an antigen that does not itself induce production of antibodies harmful to the recipient individual. Carriers are slowly metabolized macromolecules including, without limitation, inactive virus particles, proteins, polysaccharides, polyglycolic acids, amino acid copolymers, and like carriers well known in the art.

“Adjuvants” are used to enhance efficacy of the composition and include, but are not limited to, aluminum hydroxide (alum), montanide, N-acetyl-normuramyl-L-alanyl-D-isoglutamine (the-MDP), N-acetyl-muramyl-L-threonyl-D-isoglutamine (nor-MDP), and the like adjuvants known in the art.

“Pharmaceutically acceptable vehicle” refers to the water, saline, glycerol, ethanol, etc. used for dissolution, suspension, or mixing of components in the pharmaceutical composition.

The term “manipulation” of the mosquito immune system by Pfs47 can mean, without wishing to be bound by theory, the suppression, evasion, or other avoidance of the mosquito's normal immune system mechanism that usually enables destruction or substantial reduction of the parasite.

The present invention contemplates a pharmaceutical composition comprising P47 protein, an immunogenic fragment thereof, a variant of either the protein or the immunogenic fragment, or a mixture thereof. The full length P. falciparum surface protein P47 (Pfs47) and P. vivax surface protein P47 (Pvs47) protein used in the present invention is set forth in SEQ ID NO:1 and SEQ ID NO:2, respectively. The immunogenic fragment necessarily must not be missing any sequence essential to the formation or retention of an epitope. Likewise, the variant sequence must retain sequences necessary for the desired function or effect on the intended target. The P47, the immunogenic fragment, or the variant can include other sequences that do not block or prevent the formation of the epitope of interest or other functional sequence. The pharmaceutical composition can additionally include other human pathogen antigens that are useful in eliciting an immune response, including influenza, measles, mumps, diphtheria, tetanus, pertussis, poliovirus, hepatitis B virus, varicella, N. meningitides, and rubella.

The “immunogenic fragment” has a length of at least five amino acids. With increasing preference, the length of the immunogenic fragment is at least 7, 9, 11, 13, 15, 17 and 19 amino acids. The fragment can generate antibodies that block malaria transmission.

A “variant” of P47 or its immunogenic fragment is a polypeptide having at least about 80% identity to either P47 protein (Pfs47 or Pvs47) or an immunogenic fragment thereof, and which retains the function of being immunogenic. In increasingly preferred embodiments, the variant has at least 85%, 90%, 95%, 98%, or 99% identity to the P47 protein or an immunogenic fragment thereof.

The invention also comprises a recombinant polynucleotide comprising a nucleotide sequence encoding an immunogenic fragment of the P47 protein, a variant of the protein or the immunogenic fragment. The P47 fragment or variant can be, e.g., Pfs47 or Pvs47. The immunogenic fragment is at least about five amino acids long. The variant encoded by the nucleotide sequence has at least about 80% identity to the immunogenic fragment.

In another embodiment, the invention encompasses a vector comprising the polynucleotide that comprises a nucleotide sequence encoding a P47 immunogenic fragment or a variant of the P47 immunogenic fragment. Appropriate vectors including plasmid and viral vectors are known in the art. The vector can further be used to transfect a host cell using methods known in the art. An expression vector, such as the VR1020 plasmid vector, can be used to immunize animals or to express P47 in vertebrate cells, such as human kidney cells and obtain recombinant protein.

The P47 protein, its immunogenic fragment, or variant can be made by any method that provides the desired epitope or functional sequence. A preferred method is recombinant expression in E. coli to provide non-glycosylated antigens in native conformation. This is particularly useful because natural Pfs47 is not glycosylated. Alternatively, P47 can be expressed using the baculovirus system and glycosylation can be chemically removed from the recombinant protein.

In another embodiment, the P47 protein, its immunogenic fragment, or its variant can be modified as appropriate to enhance properties such as in vitro stability and the like, or in vivo properties including its pharmacokinetics.

The pharmaceutical compositions of the subject invention can be prepared by known methods of combination of compounds in admixture with a pharmaceutically acceptable carrier. Suitable carriers and their formulation with proteins are described in Remington's Pharmaceutical Sciences (16^{th} ed. Osol, E. ed., Mack Easton Pa. (1980)).

The subject invention also comprises a method of substantially inhibiting or substantially reducing Plasmodium parasite transmission by administration to vertebrates of a pharmaceutical composition comprising P47 protein, its immunogenic fragment, or a variant thereof. The composition can also include a pharmaceutically acceptable carrier, an adjuvant, and/or a pharmaceutically acceptable vehicle.

The pharmaceutical composition comprising P47, its immunogenic fragment, or its variant can be used as a vaccine to block Plasmodium transmission by administration to humans and higher primates. When administered to humans, the pharmaceutical composition can additionally include other human pathogen antigens that are useful in eliciting an immune response, including influenza, measles, mumps, diphtheria, tetanus, pertussis, poliovirus, hepatitis B virus, varicella, N. meningitides, and rubella.

Methods of administration of a pharmaceutical composition of the subject invention to a human or higher primate can be carried out by any suitable means, including intravenously, intramuscularly, intranasally, and subcutaneously.

The subject invention also encompasses a pharmaceutical composition for substantially blocking or substantially reducing transmission of a Plasmodium parasite in vertebrates, wherein the composition comprises an antibody or a fragment thereof, which is specifically reactive to P47 or an immunogenic fragment or variant thereof, and a pharmaceutically acceptable carrier. The antibody can be a monoclonal antibody or a polyclonal antibody. The parasite can be, e.g., P. falciparum or P. vivax.

The invention further comprises a method of substantially blocking or substantially reducing transmission of a Plasmodium parasite in a population of humans or higher primates comprising administering, to at least one human or higher primate, a pharmaceutical composition comprising antibodies or fragments thereof, which are specifically reactive to P47, or immunogenic fragments or variants thereof. The composition can further include a pharmaceutically acceptable carrier.

The composition used in the foregoing method can further include one or more antigens of a human pathogen, including, without limitation, influenza, measles, mumps, diphtheria, tetanus, pertussis, poliovirus, hepatitis B virus, varicella, N. meningitides, and rubella. The composition can further include an adjuvant.

The method of administering the pharmaceutical composition comprising the antibody or related compounds includes methods known in the art, including, without limitation, intravenous, intramuscular, intranasal, and subcutaneous.

In another embodiment, the invention comprises a method of providing one or more antibodies or fragments or variants thereof, specifically reactive to one or more antigens, to a mosquito, comprising administering to a human a composition comprising the one or more antigens. The antigen can be P47 protein or an immunogenic fragment or variant thereof. The P47 can be, e.g., Pfs47 derived from P. falciparum or Pvs47 derived from P. vivax.

In another embodiment, the invention comprises a recombinant plasmid comprising a polynucleotide that encodes P47 protein (or an immunogenic fragment or variant thereof), or an antibody (or antibody fragment) specific to P47 (or its immunogenic fragment or variant). The translation of the polynucleotide can be under the control of a viral promoter such as the Rous Sarcoma Virus or Cytomegalovirus. The plasmid can also include a polyadenylation signal such as the bovine growth hormone or rabbit beta-globulin polyadenylation sequences. The plasmid can be used in a method of treating mammals comprising administering a pharmaceutically effective amount of the recombinant plasmid to an individual in need thereof.

The invention further comprises a method for identifying pharmaceutical agents or drugs that can interfere with the function of P47. Generally, this method involves producing an infected mosquito population by contacting or feeding the mosquito population with a blood meal comprising Plasmodium gametocytes using, e.g., a MFA. The candidate pharmaceutical agent can be added to the gametocyte culture to determine whether the candidate molecule affects the capacity of the mosquitoes to substantially reduce the Plasmodium parasite transmission. Those candidate molecules determined to improve the mosquitoes' capacity to reduce the transmission of Plasmodium are identified as pharmaceutical agents. Large numbers of candidate agents can be pre-screened for potential interference with P47 using cell lines specific for Pfs47 or Pvs47.

In the foregoing method, the Plasmodium parasites can be provided in P. falciparum gametocyte culture or as blood from an infected donor. Further, contacting of P. falciparum with mosquitoes can be managed in a MFA. The candidate molecules can also be contacted with the infected mosquito population using the MFA.

The invention comprises further methods for identifying pharmaceutical agents that can be useful as vaccines. In one such method, the pharmaceutical agents can restore JNK signaling in a system in which inactivation of the JNK signaling pathway has been established. The system is contacted with a candidate agent, and the effect of the candidate agent on the restoration of JNK signaling is determined. In one aspect, the system comprises Drosophila S2 cells or other insect cell lines in which the intact JNK signaling pathway has been inactivated by exposing the cells to P47. The process by which the Drosophila S2 cells or other insect cells can have their intact JNK signaling pathway inactivated may be by, e.g.: a) exposing the surface of said cells to P47 by adding recombinant P47 protein to the culture media; and/or b) transfecting the cells with an expression plasmid whereby the recombinant P47 is expressed in the cytoplasm of the cells. The determination of whether the candidate agent has restored JNK signaling in the Drosophila S2 or other insect cell line is accomplished by measuring JNK phosphorylation or by using a reporter gene, such as green fluorescent protein (GFP).

In an alternate embodiment, the method for identification of pharmaceutical agents that can be useful as vaccines uses, as an assay system, mosquitoes that have been infected with Plasmodium parasites that express P47, resulting in the JNK signaling pathway of the mosquitoes being inhibited. This, in turn, results in a lack of activation of the mosquito complement-like system. In this embodiment, a candidate agent that restores JNK signaling can be evident by the mosquitoes' capacity to substantially block parasite infection. In one aspect, the mosquitoes are A. gambiae or other anopheline mosquito, and the Plasmodium parasites are P. falciparum or P. vivax.

The subject invention also comprises a transgenic mosquito that comprises cells that express an inhibitory factor that interferes with the function of P47. This inhibitory factor can be an exogenous polynucleotide sequence that encodes an antibody specifically directed to P47 and that interferes with the immuno-suppressive activity of P47. Antibodies, polyclonal or monoclonal, can be obtained using methods known in the art.

In a further embodiment, the transgenic mosquito is paratransgenic, and bacteria native to the mosquito gut microbiota are transfected with a polynucleotide encoding the inhibitory factor. The symbiont bacteria of the gut microbiota can be readily transfected with, e.g., plasmids containing the exogenous polynucleotide, are grown easily in vitro, and can export the exogenous polypeptide. The engineered bacteria remain stable and are easily delivered to the gut of the mosquito, where they continue to export exogenous polypeptide. The exogenous polypeptide cannot be toxic to the symbiont bacteria or the mosquitoes. By this mechanism, the paratransgenic mosquitoes can disrupt the inhibitory function of Plasmodium Pfs47, permitting the mosquitoes' immune system to recognize the Plasmodium parasites and substantially reduce their transmission.

For example, suitable symbionts for mosquitoes, such as A. gambiae, include acetic acid bacteria (AAB), especially members of Acetobacter and Gluconacetobacter genera, and Pantoea agglomerans (P. agglomerans). Acetic acid bacteria and P. agglomerans have been found to be native symbionts of mosquitoes. AAB bacterium Asaia spp. has been found to be “a dominant bacterium within the insect microbial community,” including Anopheles (An.) stephensi, An. maculipennis, An. gambiae, and Aedes aegypti. The predominant habitat of the AAB in mosquitoes has been found to be the gastrointestinal tract, which ensures access to diet-derived sugars. The gastrointestinal tract is acidic, aerobic, and provides a sugar diet, thereby permitting growth and reproduction of the AAB. The AAB are spread naturally through the host mosquito population by vertical and horizontal transmission routes. (Crotti, E. et al. (2010) Appl. Environ. Microbiol. 76(21):6963; Wang S. et al. (2012) Proc Natl Acad Sci USA 109:12734-9).

The inhibitory factor employed in the paratransgenic mosquito can be an exogenous polynucleotide sequence that can, for example, encode an antibody specifically directed to P47 that interferes with the immuno-suppressive activity of P47. Again, the antibody to P47, polyclonal or monoclonal, can be obtained by methods known in the art.

It is also contemplated that the invention includes a method of detection or determination of malaria antibodies in a biological sample. The method uses P47 (or its immunogenic fragment or variant), preferably purified or essentially purified, and is typically performed in vitro. The detection or determination of malaria antibodies in the biological sample of an individual can be for purposes of diagnosis, or for monitoring of response to malaria treatment and prognosis for patients previously diagnosed.

The method includes the steps of providing or obtaining a biological sample from an individual who may have malaria and antibodies to P47; contacting the sample with a P47 protein or an immunogenic fragment under conditions which allow the formation of an immune complex; and detecting the presence of the complexes of P47 protein or fragment and the antibody.

The assay methods use conditions that allow the P47 (or its fragment) to bind to antibody in the biological sample. These conditions include physiologic pH, temperature, and ionic strength with an excess of P47 (or fragment), followed by incubation.

The contacting step of the method can be carried out in solution. In this embodiment, the P47 or its fragment can be linked to a detectable label. The label can be, for example, fluorescent, chemiluminescent, radioactive, or dye molecules. Labeled complex can be detected by known methods including fluorimetry, chemiluminescence, radiometry, or colorimetry. In another aspect, during incubation, the complex of antibody and antigen may immunoprecipitate.

In the alternative, the contacting step of the method can be carried out with the P47 immobilized to a solid support. The P47 may be labeled, e.g., with a fluorescent label. Examples of solid supports include, without limitation, nitrocellulose (in membrane or microtiter well form), polyvinylidine fluoride, polyvinyl chloride, polystyrene latex, activated beads, and the like. This method may further comprise a step of removing unbound components after complex formation on the support. The detection of antibody complexed to the P47 can be accomplished using methods known in the art, including, without limitation, fluorimetry.

The invention further comprises a kit for detecting or determining the presence of malaria antibodies in a biological sample. The kit includes a P47 protein, its immunogenic fragment, or a variant thereof; a buffer for enabling immune complex formation between the P47 protein, fragment, or variant, and antibodies against malaria present in a biological sample. In one aspect, the P47, fragment, or variant is immobilized on a solid support (e.g., ELISA plate). There may also be included a wash solution to remove uncomplexed components.

All references cited herein are incorporated herein in their entirety by reference.

### EXAMPLES

### Example 1

### Determination that Pfs47 is Critical for P. falciparum to Evade the Immune System of A. gambiae Mosquitoes

It was discovered that Pfs47 is a key factor for survival of P. falciparum parasites in the mosquito A. gambiae, a major natural vector of human malaria in Africa. Pfs47 allows the parasite to infect the mosquito without activating the mosquito immune system. The genomic region responsible for the immune evasion by using classic QTL analysis with the progeny of a cross between Brazilian 7G8 X African GB4 parasites was mapped. The genomic location of the immune evasion gene was confirmed by linkage group selection analysis of individual oocysts from the recombinant population from the cross. These experiments defined a 171 kb region that codes for 41 genes. The expression of each of these genes in gametocytes and ookinetes and sequenced their coding regions from both strains was determined. Based on this analysis, two top candidate genes, Pfs47 and Pfs48/45, which have 4 and 2 non-synonymous polymorphisms, respectively, and encode for proteins known to be expressed on the surface of the sexual stages of the parasite, were defined.

Pfs47 and Pfs48/45 are expressed in gametocyte stages of Plasmodium and have been investigated as candidates for transmission-blocking vaccines. Knockout (KO) lines for these two genes were generated by Drs. Sauerwein and Eling from the Radboud University Nijmegen Medical Center and collaborators in the African NF54 P. falciparum Strain, and their effect on Plasmodium infectivity to mosquitoes has been published (van Dijk et al., 2001; van Schaijk et al., 2006). It was confirmed that Pfs48/45 KO parasites infect mosquitoes at low levels due to deficient fertility. But the parasites that infect the mosquito are able avoid destruction by the mosquito immune system, indicating that this gene is not mediating immune evasion by the parasite. As previously reported, Pfs47 KO parasites (in a NF54 genetic background) do not have a fertility problem and form ookinetes that invade the A. stephensi mosquito midgut in high numbers. However, it was found that when Pfs47 is no longer expressed, the parasites no longer survive in the A. gambiae refractory strain, suggesting that they can no longer evade the mosquito immune system. This was confirmed by silencing expression of the mosquito immune factor TEP1, a key effector of the mosquito antiplasmodial response. When TEP1 expression was silenced, the Pfs47 KO parasites were no longer eliminated, indicating that the parasites are actively killed by the mosquito. Furthermore, TEP1-mediated killing of Pfs47 KO parasites was also observed in the A. gambiae G3 strain, which is highly susceptible to infection with wild type NF54 P. falciparum parasites.

### Example 2

### Pfs47 Gene Mediates Evasion of the Mosquito Immune System

It was discovered that Pfs47 is a key factor for survival of P. falciparum parasites in the mosquito A. gambiae, a major natural vector of human malaria in Africa. A combination of genetic mapping, linkage group selection, and functional genomics was used to identify Pfs47 as a P. falciparum gene that allows the parasite to infect A. gambiae without activating the mosquito immune system. Disruption of Pfs47 greatly reduced parasite survival in the mosquito and this phenotype could be reverted by genetic complementation of the parasite or by disruption of the mosquito complement-like system. Pfs47 suppresses midgut nitration responses that are critical to activate the complement-like system. Direct experimental evidence was provided that immune evasion mediated by Pfs47 is critical for efficient human malaria transmission by A. gambiae. The A. gambiae L3-5 strain was selected to be refractory (R) to Plasmodium cynomolgi (simian malaria), but also eliminates most other Plasmodium species including P. falciparum strains from the New World, and forms a melanotic capsule (i.e., deposition of melanin, a black insoluble pigment) around the dead parasites.

In contrast, this strain is highly susceptible to infection with some African P. falciparum strains, such as NF54, 3D7, and GB4. Some parasite lines from malaria-endemic areas where A. gambiae is the natural vector are able to evade the mosquito immune system. Co-infection experiments reveal that the immune response (or lack thereof) to a P. falciparum strain did not affect the fate of other parasites present in the same mosquito midgut; suggesting that parasite survival is determined by genetic differences between P. falciparum strains. In this study, quantitative trait locus (QTL) mapping, linkage group selection, and functional genomics were used to identify the first P. falciparum gene that promotes infection by modulating the host immune system.

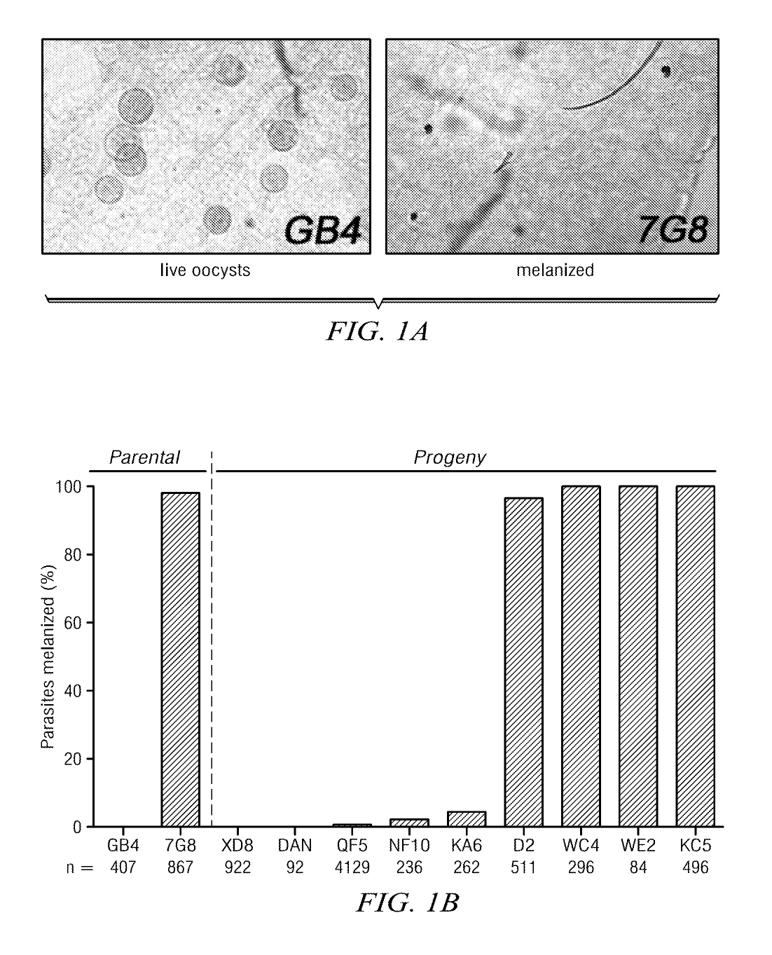

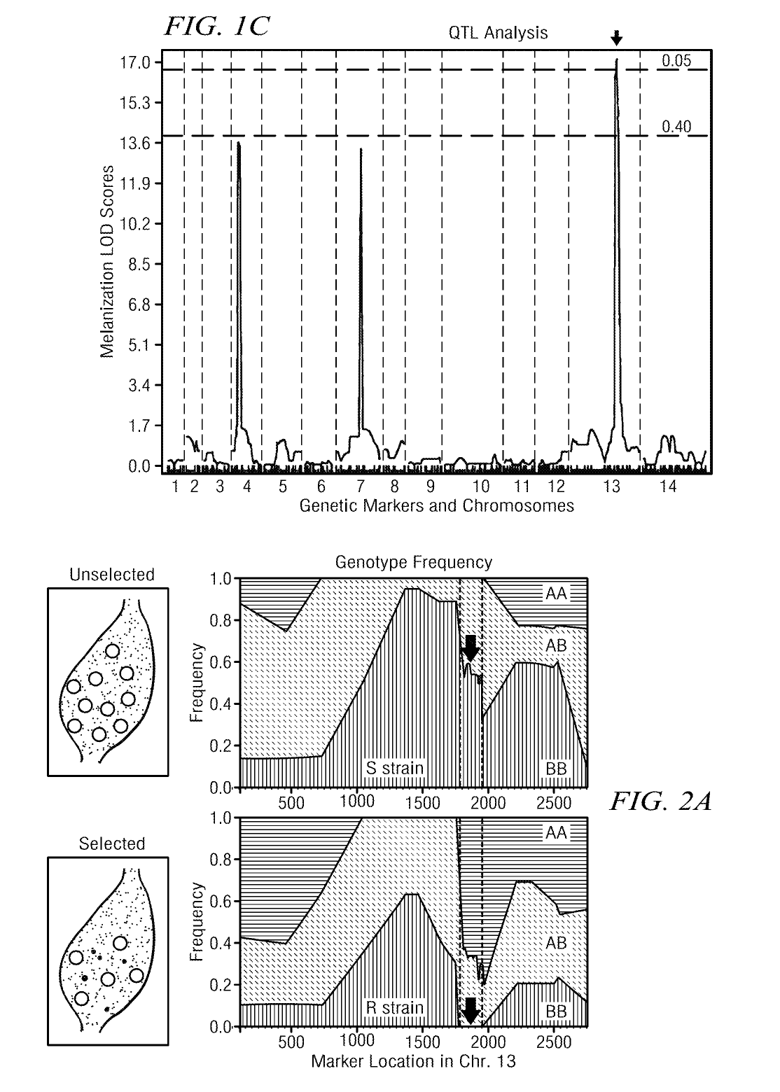

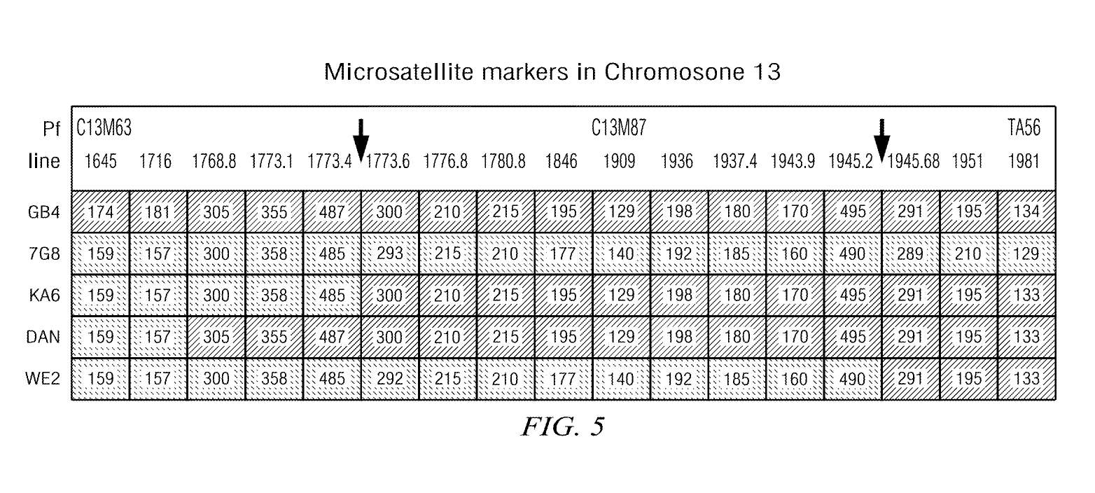

There is the phenotypic difference between A. gambiae R infected with two P. falciparum lines—7G8 from Brazil (97-100% melanized) and GB4 from Ghana (0-3% melanized) (FIG. 1A)—that have been previously subjected to a genetic cross. Nine cloned progeny lines were phenotyped. Five of them had the GB4 phenotype and survived well (0-5% melanization), while four had the 7G8 phenotype and were mostly melanized (98-100% melanization) (FIG. 1B). The presence of two distinct phenotypes in the progeny suggested a monogenic trait. Repeated attempts to phenotype 16 additional progeny lines failed, because most clonal lines had lost the ability to generate mature gametocytes. QTL mapping, using a previously reported linkage map and the phenotypes obtained, identified three logarithm of odds (LOD) peaks (FIG. 1C), but only one of them, located in chromosome 13 (Chr13), was significant (P<0.05). The boundaries of the recombination sites for this locus were precisely mapped and defined a 172-kb region coding for 41 genes (FIG. 5; Table 1).

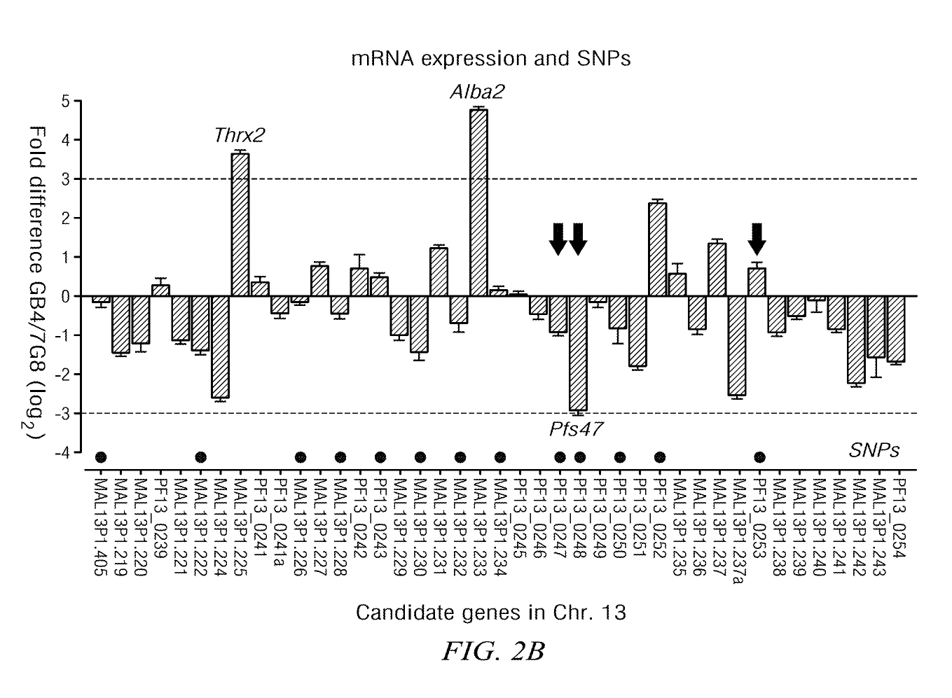

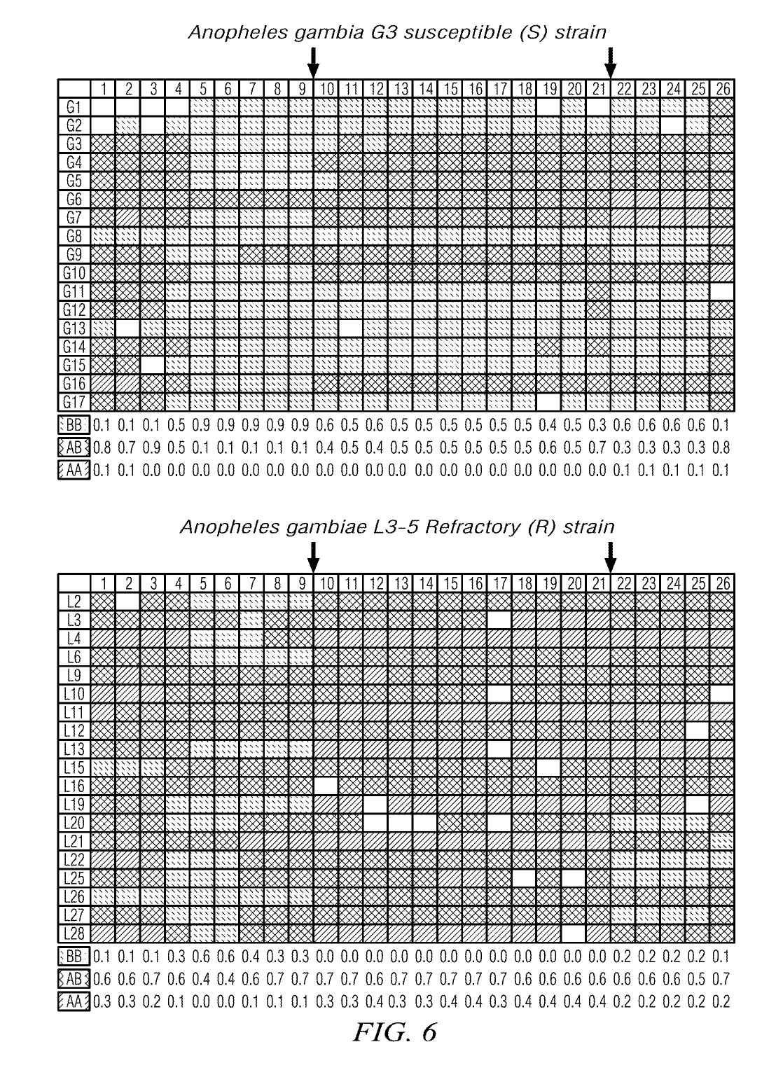

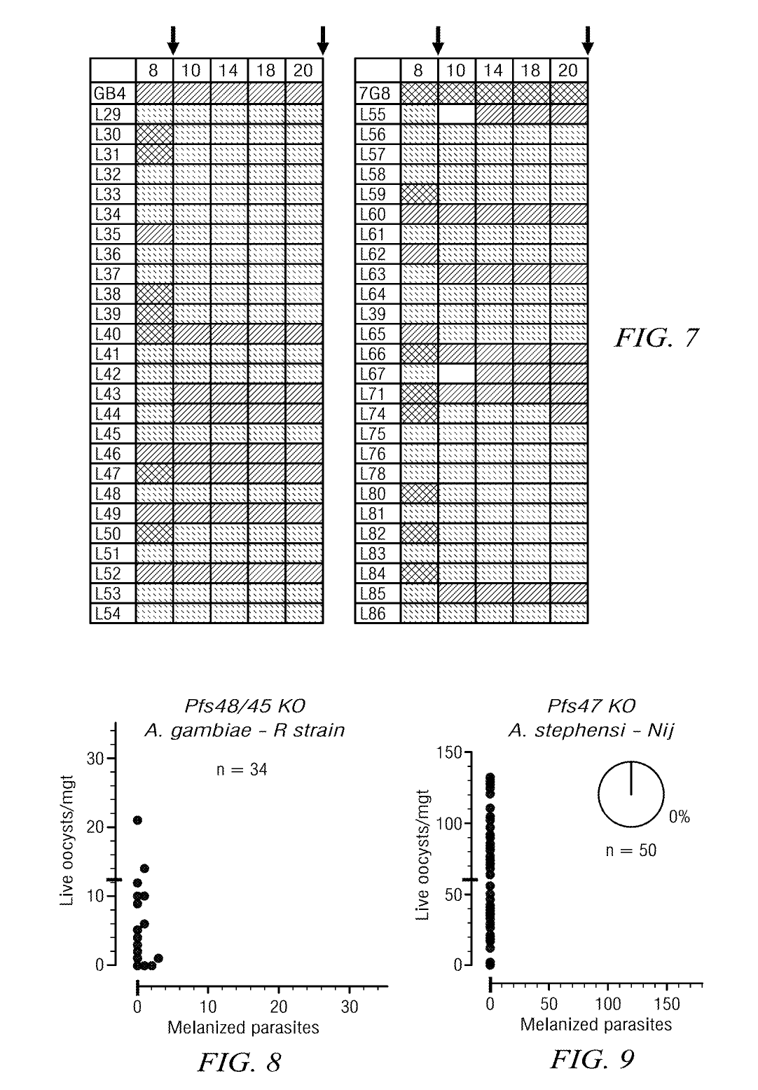

Linkage group selection analysis, a method that allows de novo location of loci encoding selectable phenotypes of malaria parasites, was used to obtain independent confirmation of the locus in Chr13. The un-cloned recombinant progeny from the original genetic cross was used to generate gametocytes and infect either the R strain or a permissive susceptible (S) A. gambiae G3 line in which both parental parasite lines survive. Individual oocysts were isolated, subjected to whole genome DNA amplification, and genotyped for multiple markers along Chr13. Oocysts derive from the diploid ookinete stage and can be homozygous for the African GB4 (AA) or Brazilian 7G8 alleles (BB), or heterozygous (AB). In the S strain, the BB genotype is highly abundant in the central region of Chr13, reaching a frequency of >90% (FIG. 2A; FIG. 6, Table 2), which was already observed in the progeny clones from the genetic cross and is not due to selection by the mosquito, because both parental strains survive in the S strain. In the R strain, a well-defined region was identified, indicated by the dotted line, in which the BB genotype is under strong negative selection and is totally absent (0%) (FIG. 2A). In contrast, prevalence of the BB genotype in the same chromosomal region is 55% in the S strain (P<0.00001; χ² test), which does not exert selective pressure on the parasite. It is noteworthy that the two markers that define this region (FIG. 2A, dotted lines) are the same as those that limit the 172-kb region identified by QTL analysis. Although 50 additional individual oocysts dissected from the R strain were genotyped, no oocyst with the BB genotype was detected for any of the markers within the region under strong selection (FIG. 7). The locus could therefore not be narrowed down any further.

Gene expression analysis of the 41 candidate genes in the ookinete stage identified three genes with large differences in expression (8-fold or higher) between the parental lines: Pfs47 (PF13_{—}0248), thioredoxin 2 (MAL13P1.225), and the nucleic acid binding protein ALBA2 (MAL13P1.233) (FIG. 2B; Table 3) (P<0.0001; t-test). Thioredoxin 2 and ALBA2 also had differences in expression between the parental lines in the gametocyte stage (Table 3). Sequencing the coding regions of the 41 candidate genes identified non-synonymous single nucleotide polymorphisms (SNPs) between the parental lines in 13 genes (FIG. 2B, magenta dots; Table 4). Some non-synonymous SNPs in three of these genes, four SNPs in Pfs47, and one in Pf48/45 (PF13_{—}0247) and ethanolamine phosphate cytidylyltransferase (PF13_{—}0253) also correlate with parasite survival in two other P. falciparum strains (FIG. 2B, black arrows; Table 4). The GB4 SNP alleles are shared with the NF54 and 3D7 strains that also survive, and the 7G8 SNP alleles with the SL strain that is melanized by the R strain. Five genes were selected as top candidates for detailed genetic analysis based on large differences in gene expression and/or on polymorphisms that correlates with survival in other strains (Table 5).

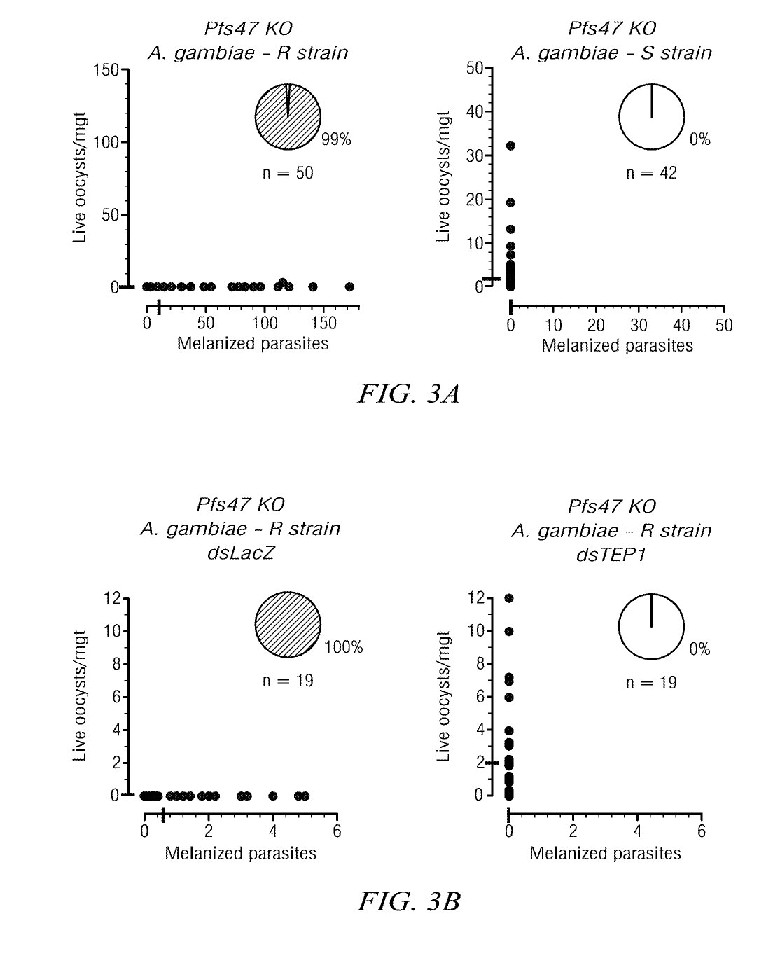

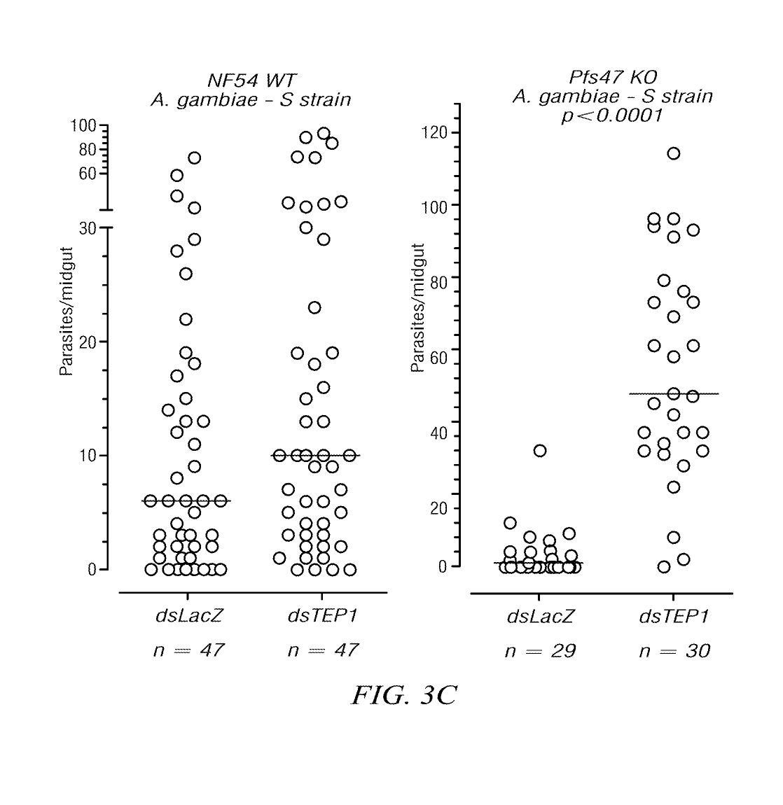

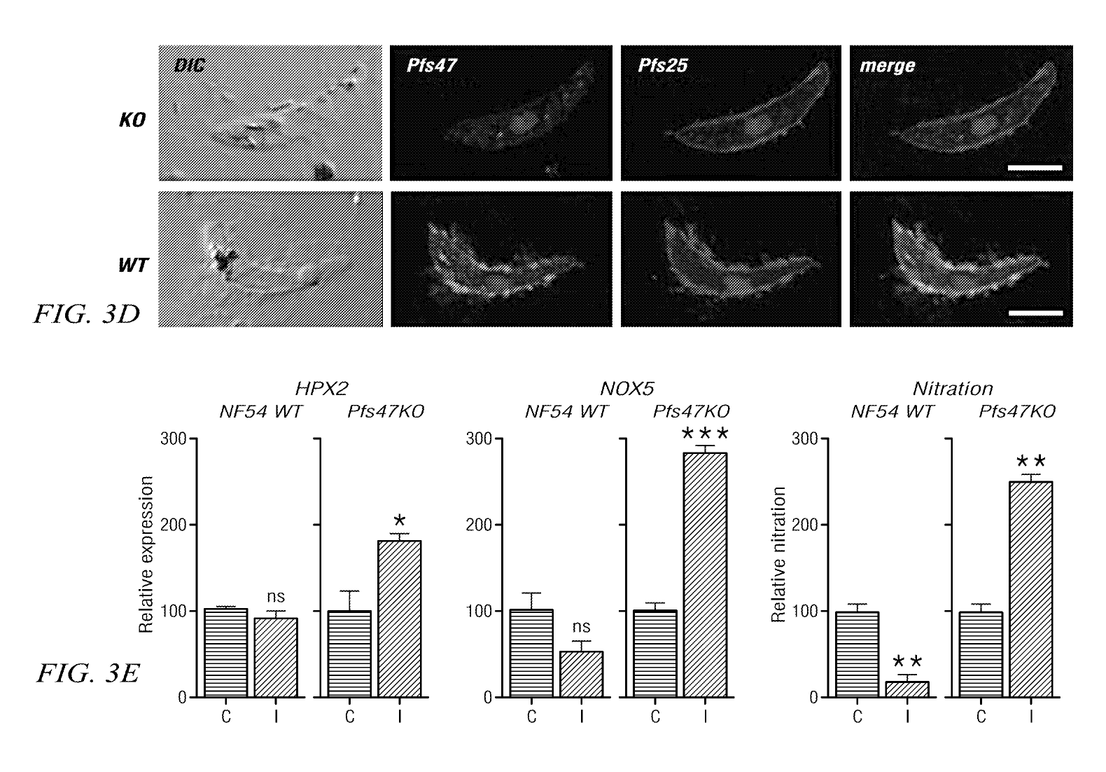

Two top candidate genes, Pfs47 and Pfs48/45, code for members of the 6-cysteine protein family that are expressed on the gametocyte surface. Previous gene disruption experiments in the NF54 line revealed that Pfs48/45 is critical for gamete fertility. Pfs47 is expressed in female gametocytes but is not essential for P. falciparum fertilization, although its homolog in Plasmodium berghei is required for female gamete fertility. The intensity of infection with the Pfs48/45 knockout (KO) line (NF54 genetic background) in the R strain was low, probably due to reduced fertility, but those parasites that invaded the midgut had a similar phenotype as wild-type (WT) NF54 parasites and only 3% were melanized (FIG. 8); indicating that Pfs48/45 is not required to evade the mosquito immune system. In contrast, Pfs47 KO (NF54 genetic background) parasites develop and invade the midgut but are eliminated by R mosquitoes (99% melanization) (FIG. 3A); however, although Pfs47 KO parasites are not melanized by S mosquitoes (FIG. 3C), the infection level in the S strain (median of 1 oocyst/midgut) is much lower than that in An. stephensi mosquitoes (60 oocysts/midgut median) (FIG. 9). Notably, this An. stephensi strain has been selected to be highly permissive to P. falciparum infection.

To determine whether Pfs47 interacts with the mosquito immune system, the A. gambiae complement-like system was disrupted by silencing TEP1. Reducing TEP1 expression completely reversed melanization of Pfs47 KO parasites in the R strain (FIG. 3B). In the A. gambiae S strain (G3), neither NF54 WT nor Pfs47 KO parasites were melanized (FIG. 3C); however, while TEP1 silencing had no significant effect on infection with NF54 WT parasites (FIG. 3C), it dramatically increased both the intensity (P<0.0001 Mann-Whitney test) and prevalence of infection (P<0.001; χ² test) of Pfs47 KO parasites (FIG. 3C). This indicates that Pfs47 is necessary for P. falciparum parasites to evade two well-characterized immune responses mediated by TEP1 in A. gambiae: killing followed by melanization in the R strain and parasite lysis without melanization in the S strain.

Pfs47 protein is present on the surface of WT NF54 ookinetes, the stage that invades the midgut, but is absent in Pfs47 KO parasites (FIG. 3D). The expression of HPX2 and NOX5, two enzymes that mediate midgut nitration in response to P. berghei infection and promote TEP1 activation (2) was evaluated in S mosquitoes. HPX2 and NOX5 were not induced by NF54 WT parasites and nitration levels were lower than in uninfected controls (FIG. 3E). In contrast, Pfs47 KO parasites induced expression of HPX2 and NOX5, and a robust nitration response, indicating that Pfs47 may prevent TEP1-mediated lysis by suppressing midgut epithelial nitration responses (FIG. 3E).

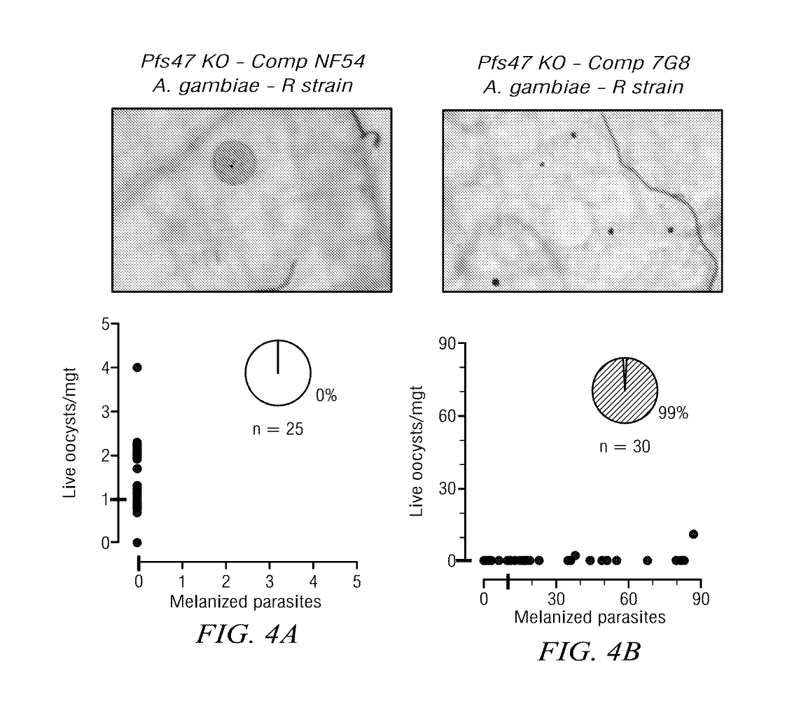

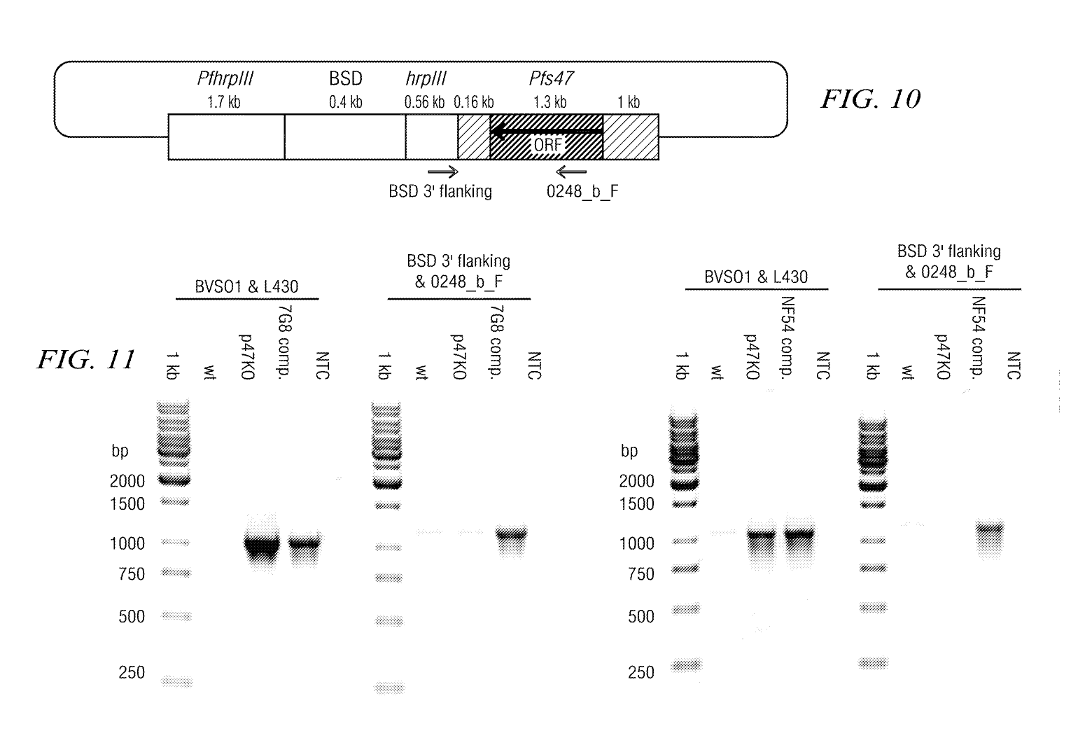

Finally, the importance of Pfs47 for parasite survival by complementing the Pfs47 KO line with different Pfs47 alleles was confirmed (FIGS. 10-13). As expected, the NF54 allele of Pfs47 reversed the melanization phenotype (0% melanization) in the R strain when the complemented parasites were kept under sustained drug pressure, confirming that this allele of Pfs47 is sufficient to evade the immune system (FIG. 4A). A reversal to a mixed live/melanization phenotype was observed when the drug pressure was reduced (FIG. 14). In contrast, complementation with the 7G8 allele failed to rescue parasites in the R strain, as 99% melanization was observed (FIG. 4B).

Together, Pfs47 was identified as an essential survival factor for P. falciparum that allows the parasite to evade the immune system of A. gambiae, a major mosquito vector in Africa. However, other parasite genes may also be involved in this process. Interestingly, Pfs47 is a highly polymorphic gene with a marked population structure in field isolates and exhibits extreme fixation in non-African regions of the world. Our findings suggest that the population structure of Pfs47 may be due to adaptation of P. falciparum to the different Anopheles vector species present outside of Africa. The fact that the 7G8 allele of Pfs47 is sufficient to evade the TEP1 complement-like system in S mosquitoes but not in the R strain indicates that there are also genetic differences in the vector that determine compatibility with parasites that express specific Pfs47 alleles. It appears that Pfs47 evolved a function in P. falciparum that increases parasite survival in A. gambiae mosquitoes and may be responsible, at least in part, for the very high rates of malaria transmission in hyperendemic regions in Africa. Disruption of the immunomodulatory activity of Pfs47 may prove to be an effective strategy to reduce malaria transmission to humans.

### Experimental Methods and Materials

### Example 2.1

### Anopheles gambiae Mosquitoes and Plasmodium Parasites

The An. gambiae L3-5 refractory strain and the Plasmodium-susceptible G3 strain were used. Mosquitoes were reared at 27° C. and 80% humidity on a 12-h light-dark cycle under standard laboratory conditions. The Plasmodium falciparum strains used—GB4, 7G8, GB4×7G8 cross progeny (cloned and un-cloned), NF54, NF54-Pfs47KO, complemented NF54-Pfs47KO and NF54-Pfs48/45KO—were maintained in O⁺ human erythrocytes using RPMI 1640 medium supplemented with 25 mM HEPES, 50 mg/1 hypoxanthine, 25 mM NaHCO₃, and 10% (v/v) heat-inactivated type O⁺ human serum (Interstate Blood Bank, Inc., Memphis, Tenn.) at 37° C. and with a gas mixture of 5% O₂, 5% CO₂, and balance N₂.

### Example 2.2

### Artificial Infection of Mosquitoes with P. falciparum and QTL Analysis

An. gambiae females were infected artificially with P. falciparum gametocyte cultures by membrane feeding. Gametocytogenesis was induced as previously described in Ifediba et al. (1981) Nature 294, 364, with 5% hematocrite, 1-2% parasetimea and in the absence of any selection drug. Mature gametocyte cultures (stages IV and V) that were 14-16 days-old were used to feed mosquitoes using membrane feeders at 37° C. for 30 min and diluting them 4-10 fold in O+ red blood cells (40% v/v in O+ human serum). Midguts were dissected 8-10 days post infection, and oocysts were stained with 0.1% (w/v) mercurochrome in water and counted by light microscopy. Infections were performed at least in duplicate. Distribution of parasite numbers in individual mosquitoes between control and experimental groups was compared using the non-parametric Mann Whitney test. Infection prevalence of mosquitoes was compared using the χ² test. Quantitative trait locus (QTL) analysis for melanization of P. falciparum in An. gambiae L3-5 was carried out with the phenotype expressed as percentage melanization of total parasites observed (live plus melanized) with the previously obtained genotypes of the P. falciparum GB4×7G8 cross progeny clones using R/QTL (22) and its interface J/QTL. The analysis was done with interval mapping assuming normal or binary distribution of the phenotype data, and both analysis gave similar results with one significant LOD peak in chromosome 13. The results that does not modify the actual experimental values and assume a normal distribution was presented.

### Example 2.3

### Microsatellite and SNP Genotyping

Microsatellite (MS) genotyping of P. falciparum clonal progeny lines was carried out by PCR amplifying MS markers using DNA from asexual P. falciparum cultures and primers described in Table S1. PCR products were run in an ABI 31000 DNA Sequencer (ABI, Fullerton, Calif.) to determine their size. Individual P. falciparum oocysts were dissected from infected midguts under the microscope using fine needles, placed in 9 μl TE buffer, and frozen at −70° C. until used. Whole-genome amplification of individual oocysts was done using GenomePlex (Sigma) following the manufacturer's protocol except for the anneal/extend step that was done at 60° C. due to the high A/T content in P. falciparum sequences. Single nucleotide polymorphism (SNP) genotyping was done with Taqman assays (ABI, Fullerton, Calif.) using primers and probes described in Table S2. Taqman PCR reaction conditions used were a step of 10 min at 95° C. followed by 50 cycles of 15 sec at 92° C. and 1.5 min at 60° C.

### Example 2.4

### Gene Expression qPCR and Sequencing

Gene expression was assessed on gametocyte cultures or 24 h post infection An. gambiae midguts (okinete stage) by extracting total RNA using the RNAeasy kit (Qiagen, Valencia, Calif.), synthetizing cDNA with the Quantitect kit (Qiagen) and SYBR green qPCR DyNamo HS (Finnzymes, Espoo, Finland). P. falciparum gene Pf10_{—}0203 (ADP-ribosylation factor) was used as internal reference with PCR primers F 5′-GATGCTGCTGGAAAAACTAC-3′ (SEQ ID NO: 217) and R 5′-CCTACATCCCATACG GTAAA-3′ (SEQ ID NO: 218). Other primers used are described in Table S6. qPCRs were performed under standard conditions using 0.5 μM of each primer with an initial denaturation step of 15 min at 95° C. and then 45 cycles of 10 sec at 94° C., 20 sec at 50° C., and 30 sec at 60° C., with a final extension of 5 min at 60° C. Gene expression of A. gambiae Hpx2 and Nox5 was assessed in a similar way using SYBR green qPCR and the A. gambiae ribosomal S7 gene as internal reference in blood-fed control, NF54 WT infected or p47KO infected mosquito midguts at 24 hours post-feeding using primers as follows: Nox5F 5′-TCATGCATCGCTACTGGAAG-3′ (SEQ ID NO: 219), Nox5R 5′-CCAGAAAAGTCCACCTTGG-3′ (SEQ ID NO: 220), Hpx2F 5′-CCGCTTCTACAACACGATGA-3′ (SEQ ID NO: 221), Hpx2R 5′-CGACCAGATGGGCAAGTAT-3′ (SEQ ID NO: 222), S7F 5′-AGAACCAGCAGACCACCATC-3′ (SEQ ID NO: 223), S7R 5′-GCTGCAAACTTCGGCTATTC-3′ (SEQ ID NO: 224). For these genes, qPCRs were performed under standard conditions using 0.5 μM of each primer with an initial denaturation step of 15 min at 95° C. and then 45 cycles of 10 sec at 94° C., 20 sec at 55° C., and 30 sec at 72° C., with a final extension of 5 min at 72° C. Sequencing of candidate genes in chromosome 13 QTL region in both GB4 and 7G8 strains was done on extracted DNA.

### Example 2.5

### dsRNA-Mediated Mosquito Gene Knockdown

Individual female An. gambiae mosquitoes were injected 1-2 day post emergence as previously described in Molina-Cruz et al. (2008) J Biol Chem 283, 3217. Briefly, mosquitoes were injected with 69 μl of a 3-μg/μl dsRNA solution 3-4 days before receiving a Plasmodium-infected blood meal. dsRNA TEP1 and LacZ were produced using the MEGAscript© RNAi Kit (Ambion, Austin, Tex.) using DNA templates obtained by PCR using An. gambiae cDNA and the primers previously described with T7 polymerase promoter sites added in the 5′-end. TEP1 gene silencing was assessed in whole sugar-fed mosquitoes by quantitative real-time PCR using primers TEP1-qF (5′-GTTTCTCACCGCGTTCGT-3′) (SEQ ID NO: 225), TEP1-qR (5′-AACCAATCCAATGCCTTCTC-3′) (SEQ ID NO: 226) and was found to be 84% lower in dsTEP1-injected mosquitoes compared with a LacZ-injected control.

### Example 2.6

### Immunostaining in P. falciparum Ookinetes

Smears from 24-28 h post-infected mosquito midguts were prepared on poly-L-lysine (0.01%) coated glass slides and stored at 4° C. until use. Dry smears were fixed in 4% paraformaldehyde in PBS for 1 hr at room temperature (RT) in a humidified chamber. Smears were blocked with 5% BSA in PBS for 30 min RT and rinsed with 0.01% Tween 20 for 10 min RT. Incubation with prinary antibody (rat monoclonal anti-Pfs47 antibodies 47.1, 47.2 and 47.3 or mouse monoclonal 4B7 anti Pfs25, diluted 1:500 in 1% BSA in PBS) was done overnight at 4° C. The slides were rinsed twice with 0.01% Tween 20 and once with PBS for 10 min RT. Incubation with secondary antibody diluted 1:500 in 1% BSA in PBS (Alexa Fluor-488 labeled goat anti-rat IgG or Alexa Fluor-555 labeled goat anti mouse IgG, Invitrogen) was done for 1 hr RT. The slides were rinsed as above. Smears were covered with Vectashield mounting media containing DAPI.

### Example 2.7

### Midgut Nitration Assays

Nitration assays were performed as previously described in Oliveira et al. (2012) Science 335, 856. Briefly, for each control and infected group, five midguts were dissected, fragmented, fixed with paraformaldehyde and glutaraldehyde, washed in PBS then incubated in amino triazole. Fragments were pelleted then incubated with levamisole, blocked with PBT and washed. Fragments were resuspended in PBT and divided into technical replicates each representing the equivalent of one midgut. These replicates were incubated with anti-nitrotyrosine primary antibody in PBT (1:3,000) at 4° C. overnight. Samples were washed with PBT and incubated with a secondary alkaline phosphatase-conjugated antibody (1:5,000) in PBT then incubated with ρNPP-ρ-nitrophenylphosphate and read at 405 nm in a spectrophotometer plate reader. Relative nitration was determined by normalizing the unfed control samples to 100 and represents average of 4-5 technical replicates of each of two biological replicates.

### Example 2.8

### Genetic Complementation of P. falciparum Pfs47 KO

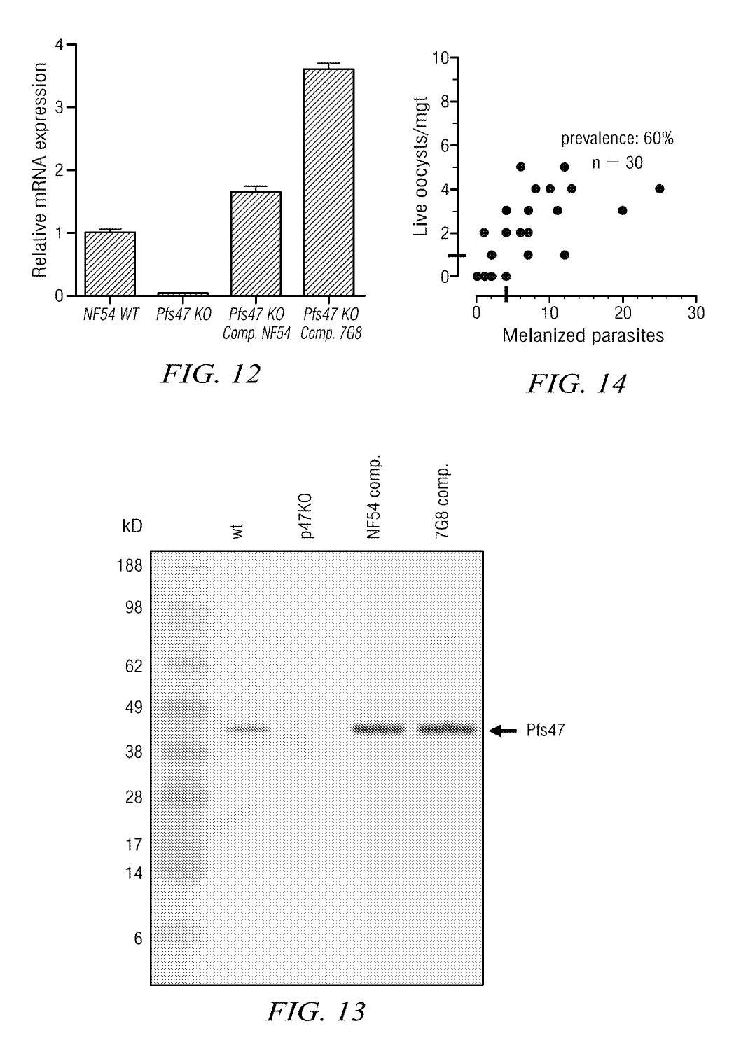

Parasite transfection was done as previously described by Deitsch et al. (2001) Nucleic Acids Res 29, 850. Briefly, 150 μl of leukocyte cleared red blood cells (RBC) (Sepacell R-500II, Fenwall) were washed once with incomplete cytomix (120 mM KCl, 0.15 mM CaCl, 2 mM EGTA, 5 mM MgCl2, 10 mM K2HPO4/KH2PO4, 25 mM HEPES, pH 7.6 adjusted with KOH) and resuspended in 400 μl cytomix. The plasmid (100 μg at a concentration of 1 μg/μl in cytomix) was added to RBC's in a chilled electroporation cuvette (Bio-Rad, 0.2 cm electrode) under sterile conditions, Electroporation was done in a Bio-Rad Gene Pulser II at 310V and 975 μF capacitance. Electroporated RBC's were washed three times with 12 ml complete culture media and mixed with P. falciparum NF54-Pfs47KO schizonts purified by Percoll-Sorbitol gradient. Culture media was changed daily and selection drugs (10 μM pyrimethamine and 4 μM BSD) were added once the cultures reached 6% parasitemia and maintained continuously in the asexual cultures unless stated otherwise. The gametocyte cultures were done without the selection drugs. P. falciparum NF54-Pfs47 KO was prepared as previously described in Van Schaijk et al. (2006) Mol Biochem Parasitol 149, 216. Plasmids containing Pfs47 alleles from 3D7 (a clonal line obtained from NF54) or 7G8 and their presumed endogenous 5′ promoter region (1,030 bp upstream of the start ATG) and 3′ UTR (162 by downstream of stop codon) were prepared by amplifying Pfs47 from P. falciparum 3D7 or 7G8 lines using the following primers: Pfs47_inf_F:5′-AGCTGGAGCTCCACCGCGGTTTATAAAAACATTCCTAACACATT-3′(SEQ ID NO: 227) and Pfs47_inf_R: 5′-CGGGGGATCCACTAGTATTTACCTTACATTTATCTCCA-3′ (SEQ ID NO: 228) (the sequence directed to Pfs47 is indicated in bold characters, the rest of the sequence is complementary to the plasmid used for In-Fusion cloning). The PCR product included noncoding regions upstream (1 kb) and downstream (0.16 kb) of the Pfs47 open reading frame (ORF). The PCR product was cloned using the In-Fusion HD cloning kit (Clontech) into the previously developed pCBM-BSD plasmid linearized by restriction enzyme digestion with SacI and SpeII (New England Biolabs) (FIG. 10). Plasmid purification was done using Plasmid Mega kit (Qiagen) and an extra ethanol precipitation wash to resuspend the plasmid (1 μg/μl) in cytomix. The Pfs47 KO background of the complemented lines was confirmed by PCR using primers BVS01 and L430 as previously described in Van Schaijk et al. (2006) Mol Biochem Parasitol 149, 216 (FIG. 11). The presence of the pCBM-BSD plasmid with the Pfs47 insert in the complemented lines was confirmed by PCR using a primer directed to the Pfs47 coding sequence (0248_b_F 5′-AGTATGCAATAAATTCATCGTTC-3′ (SEQ ID NO: 229) and a primer directed to the pCBM-BSD backbone (BSD3′_R 5′-ATATAAGAACATATTTATTAAACTGC-3′) (SEQ ID NO: 230) (FIG. 11). Pfs47 mRNA expression in the episomally complemented Pfs47 KO lines was confirmed by qPCR on cDNA from stage IV-V gametocyte cultures (FIG. 12).

### Example 2.9

### Western Blot Analysis of Pfs47 Protein Expression

Expression of Pfs47 protein in gametocytes from different P. falciparum lines was detected by western blot (FIG. 13). Gametocytes were isolated by saponin treatment. Briefly, a volume of 30 ml of Gametocyte culture (14 day-old) was centrifuged and the pellet incubated in 5 ml PBS containing 0.08% saponin for 10 min at room temperature (RT). The isolated parasites were centrifuged, rinsed twice with PBS and frozen at −70° C. until used. The frozen pellet was resuspended in 100 ul water and 5 ul of it was mixed with NuPage LDS Sample Buffer, heated at 70° C. for 10 min, fractionated in a 4-12% NuPage Bis Tris gel (Novex), and transferred to nitrocellulose using the iBlot® dry blotting system (Invitrogen). The blot was blocked with 5% milk in Tris buffered saline with Tween 20 (0.05M Tris, 0.138M NaCl, 0.0027M KCl, pH 8; 0.05% Tween 20) overnight at 4° C., followed by incubation with a pool of anti-Pfs47 rat monoclonal antibodies 47.1, 47.2, 47.3 (1 mg/ml) diluted 1:200 in the milk solution for 2 hr at RT. Subsequently the blot was incubated for 1 hr at RT with anti-rat IgG Alkaline Phosphatese conjugate (1 mg/ml Promega) diluted 1:10,000 in milk solution. Antibody staining was detected with Western Blue stabilized substrate (Promega).

### Example 3

### Rabbit Polyclonal Antibodies to Pfs47

A polynucleotide segment encoding a Pfs47 fragment that is immunogenic can be transfected into E. coli to produce recombinant Pfs47 polypeptide in large quantities. The recombinant immunogenic polypeptide is then purified. To generate polyclonal antibodies, the purified immunogenic polypeptide or a DNA vaccine plasmid encoding the Pfs47 protein is used to immunize rabbits or mice repeatedly in order to generate polyclonal anti-serum. Serum is collected over a period of 4-6 weeks, and the quality of the antibodies is monitored by indirect ELISA. The polyclonal antibodies can be purified away from other serum proteins, if desired, using Protein A affinity chromatography.

### Example 4

### Monoclonal Antibodies to Pfs47

As described in Example 1, a polynucleotide encoding a Pfs47 immunogenic fragment is used to generate large quantities of Pfs47 immunogenic fragment. The purified immunogenic polypeptide is used to immunize mice. The spleen is isolated, and B cells from the spleen are screened to select those that produce antibodies to the Pfs47 immunogenic fragment. The selected B cells are then fused with a mouse tumor (immortal) cell line to form hybridomas. Hybridomas are screened for antibody production against Pfs47 immunogenic fragment. The selected hybridomas are then allowed to multiply in culture to produce desired monoclonal antibodies.

### Example 5

### Pharmaceutical Composition Comprising Pfs47

Essentially pure P47 protein or an immunogenic fragment thereof (see Example 3) can be admixed with adjuvant and a pharmaceutically acceptable carrier to produce a pharmaceutical composition suitable for use as a vaccine in humans using current Good Manufacturing Practices. An adjuvant can be added to enhance the immune response to the P47or its fragment. Suitable adjuvants can include a traditional adjuvant such as alum or newer adjuvants such as the Glaxo Smith Kline (GSK) ASO1 adjuvant or an experimental adjuvant such as the GLA-SE adjuvant developed by Steve Reed and Colleagues in Infectious Disease Research Institute (IDRI). Additionally, stabilizers that can increase shelf life can be added. Suitable stabilizers can include monosodium glutamate and 2-phenoxyethanol. Further, preservatives can be added so as to prevent contamination with bacteria and permit multidose vials. Suitable preservatives can include phenoxyethanol and formaldehyde.

The foregoing embodiments and examples are intended only as examples. No particular embodiment, example, or element of a particular embodiment or example is to be construed as a critical, required, or essential element or feature of any of the claims. Various alterations, modifications, substitutions, and other variations can be made to the disclosed embodiments without departing from the scope of the present invention, which is defined by the appended claims. The specification, including the figures and examples, is to be regarded in an illustrative manner, rather than a restrictive one, and all such modifications and substitutions are intended to be included within the scope of the invention. Accordingly, the scope of the invention should be determined by the appended claims and their legal equivalents, rather than by the examples given above. For example, steps recited in any of the method or process claims may be executed in any feasible order and are not limited to an order presented in any of the embodiments, the examples, or the claims.

TABLE 1
Oligonucleotide primers used for PCR amplification of microsatellite
markers along chromosome 13 of Plasmodium falciparum.

| Microsatellite | Forward Primer | Reverse Primer |
|---|---|---|
| Location (Kb) | (5′ to 3′) | (5′ to 3′) |
| 1645 | AGAGATACTATGATTATTTTA | TGTCATTATGAATGGATTCC |
| | (SEQ ID NO: 3) | (SEQ ID NO: 4) |
| 1716 | GAAACTTCTACGGGTTTCTGTA | GAAATTATACACACACGCAAAC |
| | (SEQ ID NO: 5) | (SEQ ID NO: 6) |
| 1768.8 | GACAAGTATTTCTATTTGTTAGATCA | TCGTTATATCAACAATTGCAT |
| | (SEQ ID NO: 7) | (SEQ ID NO: 8) |
| 1773.1 | CTTTGCTTACGTTTTCTTTAAATTC | TCAAGAAACCTTACAACATGATAAGA |
| | (SEQ ID NO: 9) | (SEQ ID NO: 10) |
| 1773.4 | TTCTTATCATGTTGTAAGGTTTCTTGA | TTTATATTTAACCCTTCCCAATTTTT |
| | (SEQ ID NO: 11) | (SEQ ID NO: 12) |
| 1773.6 | TTTCATTTTTGATAAAGGATAAG | ATATTTAACCCTTCCCAATTTT |
| | (SEQ ID NO: 13) | (SEQ ID NO: 14) |
| 1776.8 | AAAGCAGAATAATATGTACGATCA | GGTGGTGGTAGTAGTAGTGGTT |
| | (SEQ ID NO: 15) | (SEQ ID NO: 16) |
| 1780.8 | ATCAATAAAAATTTAACCAAGTG | TGAAAAACATTTTTGGAGTGTA |
| | (SEQ ID NO: 17) | (SEQ ID NO: 18) |
| 1846 | CCAGTTTACCAAGCTTTACG | CAGCTATTATAAATGGGGATG |
| | (SEQ ID NO: 19) | (SEQ ID NO: 20) |
| 1909 | GATGAGAGAAGGTTAAAATA | CTTCAACACATCTATGGATA |
| | (SEQ ID NO: 21) | (SEQ ID NO: 22) |
| 1936 | GACGTTCAGATTGTGTTTCC | GACAAAAACTTAACGCAAGC |
| | (SEQ ID NO: 23) | (SEQ ID NO: 24) |
| 1937.4 | GACATGATGTGTTCTGTTCATT | ATAATCCCATGAAGGATAATCA |
| | (SEQ ID NO: 25) | (SEQ ID NO: 26) |
| 1943.9 | GCATCGATAGGGATTTATGA | TCTTTGCAAATAGGAATATTGTC |
| | (SEQ ID NO: 27) | (SEQ ID NO: 28) |
| 1945.2 | TCATGTATTTGTGAAAAAGAAGCAA | TTTTGAATTAAGGAAAACATCGAC |
| | (SEQ ID NO: 29) | (SEQ ID NO: 30) |
| 1945.68 | AACAAATGAAATGAAGAGCA | ACATCAAGGTGTCCATAACA |
| | (SEQ ID NO: 31) | (SEQ ID NO: 32) |
| 1951 | TCTTTTGAAGCAGAAACGAT | CGAATTCAAGAGTTGCACTT |
| | (SEQ ID NO: 33) | (SEQ ID NO: 34) |
| 1981 | ACATCAAAATTTTATGTATC | CTCTGTGCTCTCATTGCAC |
| | (SEQ ID NO: 35) | (SEQ ID NO: 36) |

TABLE 2
Primers and probes used for microsatellite (MS) and single nucleotide
polymorphism (SNP; Taqman) genotyping of individual Plasmodium falciparum oocysts.
Marker position in chromosome 13 is indicated in Kb. The marker number in the first
column corresponds with the numbers used in Fig. 6 and Fig. 7.

| | PF Marker | | | |
|---|---|---|---|---|
| # | Location (Kb) | Primers | | Probes |
| 1 | 121.9 | F | AAATTAAACGATCACTTATTCTGTTGACAATGTT | ATTGAAGAACGTGTCCAAG |
| | Taqman | | (SEQ ID NO: 37) | (SEQ ID NO: 38) |
| | | R | TCAGGAGATATGTTCGCAAGAATCAAAA | CTTATTGAAGAACATGTCCAAG |
| | | | (SEQ ID NO: 39) | (SEQ ID NO: 40) |
| 2 | 471.9 | F | TTCCCACGTTGTAGGATAGTATACATCA | ACCTGAATCTGTGGAAAG |
| | Taqman | | (SEQ ID NO: 41) | (SEQ ID NO: 42) |
| | | R | CTGCAGCCCAATTAAATGAACTACA | AAACCTGAATCTTTGGAAAG |
| | | | (SEQ ID NO: 34) | (SEQ ID NO: 44) |
| 3 | 740.9 | F | CTTTGGAACTTTCTTCTTTGTCTTGCT | AAGTTCATCTTGGCTTAAGT |
| | Taqman | | (SEQ ID NO: 45) | (SEQ ID NO: 46) |
| | | R | CCTTTCGTACCTTTCAATATAGAGGTGTT | AAGTTCATCTTGGTTTAAGT |
| | | | (SEQ ID NO: 47) | (SEQ ID NO: 48) |
| 4 | 1057.2 | F | CAAAATGATGATCATCCTGATAATCATCATAATGATG | CTGTTAATCATCATAATTTTAATT |
| | Taqman | | (SEQ ID NO: 49) | (SEQ ID NO: 50) |
| | | R | CTTTTCTTTGTGTGGGATCGTTTGA | CTGTTAATCATCATAATTTTAATT |
| | | | (SEQ ID NO: 51) | (SEQ ID NO: 52) |
| 5 | 1370 | F | GGGTTATTGGTAAGCATGGAATTAAAATAAATTTTACT | CCACGTTCTTGCCAGAAG |
| | Taqman | | (SEQ ID NO: 53) | (SEQ ID NO: 54) |
| | | R | CAGTGGTTTCATGATTAAAATTATGTTGTATAGCT | CCACGTTCTTACCAGAAG |
| | | | (SEQ ID NO: 55) | (SEQ ID NO: 56) |
| 6 | 1466.2 | F | CCTTCTCTTTGTTTTTAGATGCATCACT | CAATGCAAAAGAAATT |
| | Taqman | | (SEQ ID NO: 57) | (SEQ ID NO: 58) |
| | | R | GCAGTACCTCTAGCTATGAAATTGATACA | CAATGCAAGAGAAATT |
| | | | (SEQ ID NO: 59) | (SEQ ID NO: 60) |
| 7 | 1628.5 | F | GGTCCATTTGCTGCTGTGTTTAATA | ATGGAATTAGAGGCAAAAT |
| | Taqman | | (SEQ ID NO: 61) | (SEQ ID NO: 62) |
| | | R | AAACCTAATGACATAGATAAACCTCCAAATAAAAGT | ATGGAATTAGAGACAAAAT |
| | | | (SEQ ID NO: 63) | (SEQ ID NO: 64) |
| 8 | 1742.9 | F | TTGGTTCATTTGTATTGTTCATAAAAGTATTATATTTATG | CAAACTTCTTTTTGTATTGTC |
| | Taqman | | (SEQ ID NO: 65) | (SEQ ID NO: 66) |
| | | R | ATGATGAAAAGAAGAAATTAAATGTATCTGATAGAAATATACCT | CAAACTTCTTTTTTTATTGTC |
| | | | (SEQ ID NO: 67) | (SEQ ID NO: 68) |
| 9 | 1746.8 | F | GTGAACATAAATAGTATCACTATACTGACTTCCATT | TTGGATTCGACGTATGTAT |
| | Taqman | | (SEQ ID NO: 69) | (SEQ ID NO: 70) |
| | | R | GGATGATGTTTCATTAAAAAGCAAAAAATTACTATTTCC | TTGGATTCGACATATGTAT |
| | | | (SEQ ID NO: 71) | (SEQ ID NO: 72) |
| 10 | 1799.8 | F | CAGATGAAGAGGATAAAGATGATGATGATAATGATAA | CATTTCTATCTTTTGGTTCTAT |
| | Taqman | | (SEQ ID NO: 73) | (SEQ ID NO: 74) |
| | | R | GAATTATATTATCCTCATCATTATGATTTATATTTTCACT | TTCATTTCTATCTTTTAGTTCTAT |
| | | | (SEQ ID NO: 75) | (SEQ ID NO: 76) |
| 11 | 1809.9 | F | GTTTATAATTCCATTATTTAAACGAACGACCTCAA | TGTTCTCTTTTTTTCGCTTCG |
| | Taqman | | (SEQ ID NO: 77) | (SEQ ID NO: 78) |
| | | R | ATTATTGTTTATGCGTCATTATATTTTTTTACATGTGATT | TTGTTCTCTTTTTTTTGCTTCG |
| | | | (SEQ ID NO: 79) | (SEQ ID NO: 80) |
| 12 | 1846 MS | F | CCAGTTTACCAAGCTTTACG | |
| | | | (SEQ ID NO: 81) | |
| | | R | CAGCTATTATAAATGGGGATG | |
| | | | (SEQ ID NO: 82) | |
| 13 | 1856.7 | F | TTTGAAAGTTTAAAAATTAAAACAGCATTTGATAC | TGGCTACAGGCGTATTT |
| | Taqman | | (SEQ ID NO: 83) | (SEQ ID NO: 84) |
| | | R | AGAACTAAACGAAATGACATACTTATATATTGGAATGT | ATGGCTACAGGTGTATTT |
| | | | (SEQ ID NO: 85) | (SEQ ID NO: 86) |
| 14 | 1865.1 | F | TCAGAGAATAGTTTAAGTTTATCAAAAAATAGTGTTTATGC | CCAAAAACAAATGATTATAG |
| | Taqman | | (SEQ ID NO: 87) | (SEQ ID NO: 88) |
| | | R | ACCCCCATCCTTCTCATCTATATATTTTACAT | CCAAAAACAAATGGTTATAG |
| | | | (SEQ ID NO: 89) | (SEQ ID NO: 90) |
| 15 | 1879.9 | F | TGTCCTTGCTATATGAATACCCAGAGA | CCTCATTATATACATCCAAGTTA |
| | Taqman | | (SEQ ID NO: 91) | (SEQ ID NO: 92) |
| | | R | GCTGTTAATTATGACAGAAAGGATACAAAAAAAAATAAAT | CCTCATTATATACATTCAAGTTA |
| | | | (SEQ ID NO: 93) | (SEQ ID NO: 94) |
| 16 | 1886.6 | F | TGTGTAAGATTTGGTGGATATCTACTGAGA | TGGTATGTTTGAAGTTTT |
| | Taqman | | (SEQ ID NO: 95) | (SEQ ID NO: 96) |
| | | R | AATGGCAAGAAAGTCATAATATCTCCGATT | TTGGTATGTTTCAAGTTTT |
| | | | (SEQ ID NO: 97) | (SEQ ID NO: 98) |
| 17 | 1895.2 | F | GCATCTTGATACTTCTTTGTATTATTATTATTATT | CAACTTCCTGGTTGTGTG |
| | Taqman | | (SEQ ID NO: 99) | (SEQ ID NO: 100) |
| | | R | GTGCCTTCTCAGAACTCTTGTCTT | TCAACTTCCTGATTGTGTG |
| | | | (SEQ ID NO: 101) | (SEQ ID NO: 102) |
| 18 | 1906.4 | F | ACAGAAGAGCAAATATATAATTCAGAATTAGGTATATCTGA | TTAGTGAACAGCAACATG |
| | Taqman | | (SEQ ID NO: 103) | (SEQ ID NO: 104) |
| | | R | TGGCATCTATTTCTATTAAGAAGGTTTTTTGGT | TAGTGAACAGAAACATG |
| | | | (SEQ ID NO: 105) | (SEQ ID NO: 106) |
| 19 | 1936 MS | F | GACGTTCAGATTGTGTTTCC | |
| | | | (SEQ ID NO: 107) | |
| | | R | GACAAAAACTTAACGCAAGC | |
| | | | (SEQ ID NO: 108) | |
| 20 | 1942.7 | F | GACTCTGATTGATGGGAAGATTTATATTATAATTCATATCA | CATTTGTTTGTTGTACATTAT |
| | Taqman | | (SEQ ID NO: 109) | (SEQ ID NO: 110) |
| | | R | CCAAGGGAAAATTTTATTCAATAATGATTCTACAGA | TTTGTTTGTTGTGCATTAT |
| | | | (SEQ ID NO: 111) | (SEQ ID NO: 112) |
| 21 | 1951 MS | F | TCTTTTGAAGCAGAAACGAT | |
| | | | (SEQ ID NO: 113) | |
| | | R | CGAATTCAAGAGTTGCACTT | |
| | | | (SEQ ID NO: 114) | |
| 22 | 2215.3 | F | GGAACCATATAAAAGTATATCAATAAATAATGTAAAAAGGA | ATTATGACAACATGGTTTAA |
| | Taqman | | AATA(SEQ ID NO: 115) | (SEQ ID NO: 116) |
| | | R | GGAGCATTCAACGTTAAAGAATTACCATT | TGACAACACGGTTTAA |
| | | | (SEQ ID NO: 117) | (SEQ ID NO: 118) |
| 23 | 2320.2 | F | GCTGAAACCGAAATTGAAGAGGAT | TCTGATGATACGAATAAT |
| | Taqman | | (SEQ ID NO: 119) | (SEQ ID NO: 120) |
| | | R | GGTGATACACTATTTAATTCATCAGAATCTTCCA | AATCTGATGATACTAATAAT |
| | | | (SEQ ID NO: 121) | (SEQ ID NO: 122) |
| 24 | 2495.5 | F | GGGATGAGCGTATAGATGAATTGGT | ATGAAACTAACGAGGTAATG |
| | Taqman | | (SEQ ID NO: 123) | (SEQ ID NO: 124) |
| | | R | TGTCCTCTACAAATTCAACACTGTTAACAT | ATGAAACTAACGACGTAATG |
| | | | (SEQ ID NO: 125) | (SEQ ID NO: 126) |
| 25 | 2525.6 | F | TCTGGAGGCAGATTTATCAAAACATTTATAGATTAT | ATTTGTACAAGGATTTAAAT |
| | Taqman | | (SEQ ID NO: 127) | (SEQ ID NO: 128) |
| | | R | TCTCGTCATTTGAATAAAAGCCACATAGA | TTGTACAAGGGTTTAAAT |
| | | | (SEQ ID NO: 129) | (SEQ ID NO: 130) |
| 26 | 2751.9 | F | TCACATAGTATGAAGATATATACAAATGAATGGAACATAAC | CCATTATGTAGAAGTCAAGATA |
| | Taqman | | (SEQ ID NO: 131) | (SEQ ID NO: 132) |
| | | R | TTTCCCACATTTTTTTTACATTCCATTTTTATAAT | CCATTATGTAGAAGTAAAGATA |
| | | | (SEQ ID NO: 133) | (SEQ ID NO: 134) |

TABLE 3
Relative mRNA expression of the 41 candidate genes in stage IV and V gametocytes
and in Anopheles gambiae L3-5 (R) midgut ookinetes 24 hours post infection
(PI) with the GB4 and 7G8 Plasmodium falciparum strains.

| | | | Expression | Expression |
|---|---|---|---|---|
| | | | Gamet. IV-V, | 24 h PI |
| Old ID | New ID | PlasmoDB 2012 annotation | GB4/7G8 | GB4/7G8 |
| MAL13P1.405 | PF3D7_1344300 | Erythrocyte membrane protein | 4.9 | 0.9 |
| | | pfemp3, putative | | |
| MAL13P1.219 | PF3D7_1344500 | Conserved Plasmodium protein, | 0.8 | 0.4 |
| | | unknown function | | |
| MAL13P1.220 | PF3D7_1344600 | Lipoyl synthase (LipA) | 0.8 | 0.4 |
| PF13_0239 | PF3D7_1344700 | Conserved Plasmodium protein, | 5.6 | 1.2 |
| | | unknown function | | |
| MAL13P1.221 | PF3D7_1344800 | Aspartate carbamoyltransferase | 1.9 | 0.4 |
| | | (atcasE) | | |
| MAL13P1.222 | PF3D7_1344900 | Conserved Plasmodium protein, | 1.9 | 0.4 |
| | | unknown function | | |
| MAL13P1.224 | PF3D7_1345000 | Conserved Plasmodium protein, | 1.0 | 0.2 |
| | | unknown function | | |
| MAL13P1.225 | PF3D7_1345100 | Thioredoxin 2 (TRX2) | 73.7 | 12.6 |
| PF13_0241 | PF3D7_1345200 | Rhomboid protease ROM6, putative | 2.8 | 1.3 |
| | | (ROM6) | | |
| PF13_0241a | PF3D7_1345300 | Conserved Plasmodium protein, | 1.7 | 0.7 |
| | | unknown function | | |
| MAL13P1.226 | PF3D7_1345400 | Conserved Plasmodium protein, | 1.2 | 0.9 |
| | | unknown function | | |
| MAL13P1.227 | PF3D7_1345500 | Ubiquitin conjugating enzyme | 4.0 | 1.7 |
| | | (UBC) | | |
| MAL13P1.228 | PF3D7_1345600 | Conserved Plasmodium protein, | 2.9 | 0.7 |
| | | unknown function | | |
| PF13_0242 | PF3D7_1345700 | Isocitrate dehydrogenase (NADP), | 1.3 | 1.6 |
| | | mitochondrial precursor (IDH) | | |
| PF13_0243 | PF3D7_1345800 | Conserved Plasmodium protein, | 2.3 | 1.4 |
| | | unknown function | | |
| MAL13P1.229 | PF3D7_1345900 | Conserved Plasmodium protein, | 1.2 | 0.5 |
| | | unknown function | | |
| MAL13P1.230 | PF3D7_1346000 | Conserved Plasmodium protein, | 0.6 | 0.4 |
| | | unknown function | | |
| MAL13P1.231 | PF3D7_1346100 | Sec61 α subunit, PfSec61 (SEC61) | 3.3 | 2.3 |
| MAL13P1.232 | PF3D7_1346200 | mog1 homolog, putative | 1.0 | 0.6 |
| MAL13P1.233 | PF3D7_1346300 | DNA/RNA-binding protein Alba 2 | 11.7 | 27.7 |
| | | (ALBA2) | | |
| MAL13P1.234 | PF3D7_1346400 | Conserved Plasmodium protein, | 1.9 | 1.1 |
| | | unknown function | | |
| PF13_0245 | PF3D7_1346500 | Conserved Plasmodium protein, | 2.8 | 1.0 |
| | | unknown function | | |
| PF13_0246 | PF3D7_1346600 | Conserved Plasmodium protein, | 0.9 | 0.7 |
| | | unknown function | | |
| PF13_0247 | PF3D7_1346700 | 6-cysteine protein (P48/45) | 3.0 | 0.5 |
| PF13_0248 | PF3D7_1346800 | 6-cysteine protein (P47) | 0.7 | 0.1 |
| PF13_0249 | PF3D7_1346900 | Conserved Plasmodium protein, | 1.6 | 0.9 |
| | | unknown function | | |
| PF13_0250 | PF3D7_1347000 | G-β repeat protein, putative | 1.3 | 0.6 |
| PF13_0251 | PF3D7_1347100 | DNA topoisomerase III, putative | 0.8 | 0.3 |
| PF13_0252 | PF3D7_1347200 | Nucleoside transporter 1 (NT1) | 9.0 | 5.3 |
| MAL13P1.235 | PF3D7_1347300 | Conserved Plasmodium protein, | 3.1 | 1.5 |
| | | unknown function | | |
| MAL13P1.236 | PF3D7_1347400 | Conserved Plasmodium protein, | 1.3 | 0.5 |
| | | unknown function | | |
| MAL13P1.237 | PF3D7_1347500 | DNA/RNA-binding protein Alba 4 | 6.4 | 2.6 |
| | | (ALBA4) | | |
| MAL13P1.237a | PF3D7_1347600 | Conserved Plasmodium protein, | 0.9 | 0.2 |
| | | unknown function | | |
| PF13_0253 | PF3D7_1347700 | Ethanolamine-phosphate | 2.5 | 1.6 |
| | | cytidylyltransferase, putative (ECT) | | |
| MAL13P1.238 | PF3D7_1347800 | Leucine-rich repeat protein | 2.1 | 0.5 |
| | | (LRR4.1) | | |
| MAL13P1.239 | PF3D7_1347900 | Conserved Plasmodium protein, | 4.5 | 0.7 |
| | | unknown function | | |
| MAL13P1.240 | PF3D7_1348000 | Conserved Plasmodium protein, | 0.7 | 0.9 |
| | | unknown function | | |
| MAL13P1.241 | PF3D7_1348100 | GTPase, putative | 1.0 | 0.5 |
| MAL13P1.242 | PF3D7_1348200 | Step II splicing factor, putative | 1.3 | 0.2 |
| MAL13P1.243 | PF3D7_1348300 | Elongation factor Tu, putative | 1.4 | 0.3 |
| PF13_0254 | PF3D7_1348400 | Conserved Plasmodium protein, | 0.6 | 0.3 |
| | | unknown function | | |

TABLE 4
Single nucleotide polymorphisms (SNPs) between the GB4 and 7G8 Plasmodium
falciparum strains in the coding regions of the 41 candidate genes linked to the
melanotic phenotype in the Anopheles gambiae L3-5 refractory strain. Also
shown are polymorphisms present in strains 3D7 which survives in the R
mosquito, and Santa Lucia (SL) which is melanized in the R mosquito.

| | Percent | | | | | DNA seq | | Santa |
|---|---|---|---|---|---|---|---|---|
| | Sequence | Confirmed | | Non | | (GB4- | 3D7 | Lucia |
| Gene ID | Covered * | SNP | Position | -Syn | GB4-7G8 | 7G8) | Allele | Allele |
| MAL13P1.405 | 50 | 1 | 1775200 | 1 | M-K | ATG-AAG | AAG (7G8) | |
| MAL13P1.219 | 100 | 1 | 1784410 | 0 | | ATA-ATC | ATA (GB4) | |
| MAL13P1.220 | 100 | 0 | | | | | | |
| PF13_0239 | 15 | 0 | | | | | | |
| MAL13P1.221 | 100 | 1 | 1793345 | 0 | | GTT-ATT | ATT (7G8) | |
| MAL13P1.222 | 25 | 2 | 1799851 | 1 | L-P | CTA-CCA | CCA (7G8) | |
| | | | 1801826 | | | AAC-AAT | AAC (GB4) | |
| MAL13P1.224 | 100 | 0 | | | | | | |
| MAL13P1.225 | 100 | 0 | | | | | | |
| PF13_0241 | 100 | 1 | 1809977 | 0 | | CAA-CGA | CAA (GB4) | |
| PF13_0241a | 0 | | | | | | | |
| MAL13P1.226 | 20 | 1 | 1816500 | 1 | T-I | ACA-ATA | ACA (GB4) | |
| MAL13P1.227 | 100 | 0 | | 0 | | | | |
| MAL13P1.228 | 100 | 1 | 1823907 | 1 | Y-I | TAT-ATT | TAT (GB4) | |
| PF13_0242 | 100 | 0 | | | | | | |
| PF13_0243 | 20 | 1 | 1829788- | 1 | GB4 | _ _ _-AAT | AAT (7G8) | |
| | | | | | deletion | | | |
| MAL13P1.229 | 100 | 0 | | | | | | |
| MAL13P1.230 | 100 | 1 | ND | 1 | GB4 | TAATAA | ___(7G8) | |
| | | | | | insertion | | | |
| MAL13P1.231 | 100 | 0 | | | | | | |
| MAL13P1.232 | 100 | 1 | 1842359 | 1 | I-V | ATA-GTA | ATA (GB4) | |
| MAL13P1.233 | 100 | 0 | | | | | | |
| MAL13P1.234 | 8 | 3 | 1852602 | 2 | D-G | GAT-GGT | GAT (GB4) | |
| | | | 1865057 | | V-I | GTT-ATT | ATT (7G8) | |
| | | | 1856731 | | | ACA-ACG | ACA (GB4) | |
| PF13_0245 | 75 | 0 | | | | | | |
| PF13_0246 | 100 | 0 | | | | | | |
| PF13_0247 | 100 | 2 | 1872932 | 2 | N-K | CTT-GTT | CTT (GB4) | GTT(7G8) |
| | | | 1872937 | | K-E | AAA-GAA | AAA (GB4) | |
| PF13_0248 | 100 | 4 | 1875819 | 4 | T-I | ACT-ATT | ACT (GB4) | ATT(7G8) |
| | | | 1875801 | | S-L | TCA-TTA | TCA (GB4) | TTA(7G8) |
| | | | 1875786 | | V-A | GTT-GCT | GTT (GB4) | GCT(7G8) |
| | | | 1875784 | | I-L | ATA-TTA | ATA (GB4) | TTA(7G8) |
| PF13_0249 | 100 | 0 | | | | | | |
| PF13_0250 | 10 | 1 | 1879940 | 1 | N-D | AAT-GAT | GAT (7G8) | |
| PF13_0251 | 100 | 0 | | | | | | |
| PF13_0252 | 100 | 2 | 1886603 | 1 | Q-E | CAA-GAA | CAA (GB4) | |
| | | | 1887195 | | | CCT-CCC | CCC (7G8) | |
| MAL13P1.235 | 25 | 0 | | | | | | |
| MAL13P1.236 | 100 | | | | | | | |
| MAL13P1.237 | 100 | 1 | 1895190 | 0 | | AAT-AAC | AAT (GB4) | |
| MAL13P1.237a | 100 | 0 | | | | | | |
| PF13_0253 | 100 | 2 | 1906178 | 2 | N-K | AAT-AAA | AAT (GB4) | AAA |
| | | | 1906387 | | K-Q | AAA-CAA | CAA (7G8) | |
| MAL13P1.238 | 100 | 0 | | | | | | |
| MAL13P1.239 | 0 | | | | | | | |
| MAL13P1.240 | 15 | 0 | | | | | | |
| MAL13P1.241 | 100 | 0 | | | | | | |
| MAL13P1.242 | 100 | 0 | | | | | | |
| MAL13P1.243 | 1 | 0 | | | | | | |
| PF13_0254 | 8 | 1 | 1942725 | 1 | A-V | GCA-GTA | GTA (7G8) | |

* All the coding regions could be amplified, but it was not possible to obtain good quality sequences for some of the PCR products, probably due to the presence of repetitive sequences.

TABLE 5
Top candidate genes in the chromosome 13 quantitative trait
locus (QTL) based on differences in expression in the ookinete
stage and single nucleotide polymorphism (SNP) analysis.

| | Expres- | Non- | Conserved |
|---|---|---|---|
| | sion | synon. | Af/Br |
| Candidate genes | GB4/7G8 | SNP | SNP * |
| Pfs47 (PF13_0248) | 0.1 | 4 | 4 |
| Pfs48/45 (PF13_0247) | 0.5 | 2 | 1 |
| Ethanolamine-phosphate | 1.6 | 2 | 1 |
| cytidylyltransferase | | | |
| (PF13_0253) | | | |
| Thioredoxin, TRX2 (MAL13P1.225) | 12.6 | 0 | 0 |
| Nucleic acid binding protein | 27.7 | 0 | 0 |
| (MAL13_P1.233) | | | |

* SNPs shared between Plasmodium falciparum GB4 and 3D7, two strains that survive in the R strain; and between 7G8 and the SL, two strains that are eliminated and melanized.

TABLE 6
Primers used for qPCR of candidate genes in the chromosome
13 quantitative trait locus (QTL).

| Gene ID | Forward Primer (5′ to 3′) | Reverse Primer (5′ to 3′) |
|---|---|---|
| MAL13P1.405 | GAATGTTCAGCTGGCGTTAT | AACAAAAACATGGACTCGTGA |
| | (SEQ ID NO: 135) | (SEQ ID NO: 136) |
| MAL13P1.219 | TTTTATACAGGTCTACTTCATTTCG | AAAGGGGAAATACACAAACAT |
| | (SEQ ID NO: 137) | (SEQ ID NO: 138) |
| MAL13P1.220 | CCGTATGTGAAGAAGCACAA | TCAGGAGGTAATGGGTTTGA |
| | (SEQ ID NO: 139) | (SEQ ID NO: 140) |
| PF13_0239 | GTCCCCATTCAGGTTTATCA | TTACAGAGGAAAAAGAAGAAAATG |
| | (SEQ ID NO: 141) | (SEQ ID NO: 142) |
| MAL13P1.221 | GCAAACTACTTAGCAGATACAACG | TGAACGTCTTCTAATCCTTCCT |
| | (SEQ ID NO: 143) | (SEQ ID NO: 144) |
| MAL13P1.222 | TGTTGTTAAAACAGATGAAGAGGA | TGCCATCACTATTTTGTTCA |
| | (SEQ ID NO: 145) | (SEQ ID NO: 146) |
| MAL13P1.224 | TTTTGGAGGTACAGGGGATT | TTCAGTGTTCTAAAATCAGGTACG |
| | (SEQ ID NO: 147) | (SEQ ID NO: 148) |
| MAL13P1.225 | CCAAGATTACAACAAAATGGATCA | GCGCTTTCCGTAATATTTTTG |
| | (SEQ ID NO: 149) | (SEQ ID NO: 150) |
| PF13_0241 | GATAGAACCAGACGCTCCAA | CATTATTATCCCCAGAAATAGGA |
| | (SEQ ID NO: 151) | (SEQ ID NO: 152) |
| PF13_0241a | CCAGCACACACTTTTGTTGA | GAAACGCCTTATTTGGACAG |
| | (SEQ ID NO: 153) | (SEQ ID NO: 154) |
| MAL13P1.226 | TCTTTTACAATATGTTCCCCTGA | CCCATGATGACATGAAACAG |
| | (SEQ ID NO: 155) | (SEQ ID NO: 156) |
| MAL13P1.227 | TGCAAATTATAGAATTCAAAAAGAG | CAATAGGTGGCTTTAAAGGAT |
| | (SEQ ID NO: 157) | (SEQ ID NO: 158) |
| MAL13P1.228 | GGTAGAAGCCAAATGTGACG | CAATAGGAGGCATGGAAGAA |
| | (SEQ ID NO: 159) | (SEQ ID NO: 160) |
| PF13_0242 | TGAAAACAAGACAATGCAAAA | AGACATGCATATGCTGATCAAT |
| | (SEQ ID NO: 161) | (SEQ ID NO: 162) |
| PF13_0243 | TATTAACATCGGGCGAAGAA | CGTTAAAGTGTTCATCATCATCC |
| | (SEQ ID NO: 163) | (SEQ ID NO: 164) |
| MAL13P1.229 | TTAATACGTGGGTTCGTTTCA | CCACAAAAGAAATATCGAGCA |
| | (SEQ ID NO: 165) | (SEQ ID NO: 166) |
| MAL13P1.230 | TCATCATATGGTAACATGGACA | TCGATAATACAAAGAGCGTTCA |
| | (SEQ ID NO: 167) | (SEQ ID NO: 168) |
| MAL13P1.231 | AATGGCAAGAAGTTGAATCG | GCACAGGCAACTAATACAAATG |
| | (SEQ ID NO: 169) | (SEQ ID NO: 170) |
| MAL13P1.232 | TTCCATGTTATTAATATATCAGCATT | GCTAAGGAAAATGGAAGTATAGAAAA |
| | (SEQ ID NO: 171) | (SEQ ID NO: 172) |
| MAL13P1.233 | AAATCGGGGGATGAAGAAG | TTCGTCCAATGGTTTCTGAT |
| | (SEQ ID NO: 173) | (SEQ ID NO: 174) |
| MAL13P1.234 | TTGTGAAATATTGAAATATGAAGC | AGGATCACAAATCCATAACTGT |
| | (SEQ ID NO: 175) | (SEQ ID NO: 176) |
| PF13_0245 | TATGCCATAGCGTTATCCAA | TTTTGTTGTCCCATTTTTGA |
| | (SEQ ID NO: 177) | (SEQ ID NO: 178) |
| PF13_246 | TGTTCTTTTTCCTTGTGTCG | TGGAGTTAAATAATCCCTTTGT |
| | (SEQ ID NO: 179) | (SEQ ID NO: 180) |
| PF13_0247 | TCAGAAGAACTTGAACCATCC | CATCTCCTTCAGCATCTTCA |
| | (SEQ ID NO: 181) | (SEQ ID NO: 182) |
| PF13_0248 | GCAGGCATTAAATGTCCATA | CTTTTGCGAATCGATTTCTT |
| | (SEQ ID NO: 183) | (SEQ ID NO: 184) |
| PF13_0249 | AACAATTCACATACCACTTACCC | TCCGCATATCTATCATTTCG |
| | (SEQ ID NO: 185) | (SEQ ID NO: 186) |
| PF13_0250 | GGTGTGTGGAAACAGGAAAT | CCATTATCATACCCAGCACA |
| | (SEQ ID NO: 187) | (SEQ ID NO: 188) |
| PF13_0251 | TTGTGATAGAGAAGGGGAACA | CAGCTGAAAACTGAGCTCTATG |
| | (SEQ ID NO: 189) | (SEQ ID NO: 190) |
| PF13_0252 | GGGTGGTTATATGTCAGCAG | ATGTTTTTCGGGAGATACGA |
| | (SEQ ID NO: 191) | (SEQ ID NO: 192) |
| MAL13P1.235 | CCGAAACGCCCTTATAAAT | AATGCCAAATCAAAACTGTCT |
| | (SEQ ID NO: 193) | (SEQ ID NO: 194) |
| MAL13P1.236 | TGGCAAGCGAAAAATATAAA | CAACCTCTTCTTCCTGCTTC |
| | (SEQ ID NO: 195) | (SEQ ID NO: 196) |
| MAL13P1.237 | ATGAATTCCCCAATTCAAAG | ATCGTGTTCCTCCAGCTAAT |
| | (SEQ ID NO: 197) | (SEQ ID NO: 198) |
| MAL13P1.237a | TTTCCTCAATATTACGGGTGA | AATTCTTTGGCATTCATGTG |
| | (SEQ ID NO: 199) | (SEQ ID NO: 200) |
| PF13_0253 | ATAAATTCGGATGAGGATGC | CATCGACCCATTTACAACCT |
| | (SEQ ID NO: 201) | (SEQ ID NO: 202) |
| MAL13P1.238 | GAACAACCCAATCTTGTTGA | TCTCTCATCCGTTTTAATTGG |
| | (SEQ ID NO: 203) | (SEQ ID NO: 204) |
| MAL13P1.239 | TGTGAGTGAGAATGGACCTG | TTTTTCAAAGTTGGACGTGT |
| | (SEQ ID NO: 205) | (SEQ ID NO: 206) |
| MAL13P1.240 | ATTAGAATACGGTGCCCTGA | TGCATAGCGAAGTATCATATCC |
| | (SEQ ID NO: 207) | (SEQ ID NO: 208) |
| MAL13P1.241 | TTGCAGTTGACTGTTGTAGGA | TGGTAAGGGGGTATGTGAAT |
| | (SEQ ID NO: 209) | (SEQ ID NO: 210) |
| MAL13P1.242 | CAAATGAAACTACCCCTAATGA | TTGAAACCCCATTTGTTTTT |
| | (SEQ ID NO: 211) | (SEQ ID NO: 212) |
| MAL13P1.243 | ATGGTCCCATAGCACAAGAG | TCGAACATTTCATCTTTGGA |
| | (SEQ ID NO: 213) | (SEQ ID NO: 214) |
| PF13_0254 | CGAAAAACAGCAGCTCAATA | CCTTTTCATCGCAGGTAGTT |
| | (SEQ ID NO: 215) | (SEQ ID NO: 216) |

1. A recombinant polynucleotide comprising a nucleotide sequence encoding an immunogenic fragment of P47 protein, or a variant thereof.

2. The recombinant polynucleotide of claim 1, wherein the fragment or variant P47 protein is a fragment or variant of Pfs47 (SEQ ID NO:1) or Pvs47 (SEQ ID NO:2).

3. The recombinant polynucleotide of claim 1, wherein the immunogenic fragment has a length of at least about 5 amino acids, or said variant has at least about 80% identity to the immunogenic fragment of P47 protein.

4. (canceled)

5. A vector comprising the polynucleotide of claim 1.

6. A host cell transfected with the vector of claim 5.

7. A pharmaceutical composition for substantially blocking or substantially reducing transmission of a parasite of the Plasmodium genus in humans, wherein the composition comprises P47 protein, an immunogenic fragment thereof, or a variant thereof and a pharmaceutically acceptable carrier.

8. The composition of claim 7, wherein the parasite is P. falciparum and the P47 protein is Pfs47 (SEQ ID NO:1), or P. vivax and the P47 protein is Pvs47 (SEQ ID NO:2).

9. The composition of claim 7, wherein the composition further comprises one or more antigens of a human pathogen.

10. The composition of claim 9, wherein the pathogen is selected from influenza, measles, mumps, diphtheria, tetanus, pertussis, poliovirus, hepatitis B virus, varicella, N. meningitides, and rubella.

11. (canceled)

12. The composition of claim 7, wherein the immunogenic fragment has a length of at least about 5 amino acids, or the variant has at least about 80% identity to the P47 protein or an immunogenic fragment thereof.

13. (canceled)

14. A pharmaceutical composition for substantially blocking or substantially reducing transmission of a parasite of the Plasmodium genus in humans, wherein the composition comprises an antibody or fragment thereof, specifically reactive to P47, or an immunogenic fragment or variant thereof, and a pharmaceutically acceptable carrier.

15. The pharmaceutical composition of claim 14 wherein the P47 is selected from the group consisting of Pfs47 from P. falciparum and Pvs47 from P. vivax.

16. The composition of claim 14, wherein the antibody is a monoclonal antibody or a polyclonal antibody.

17-18. (canceled)

19. The composition of claim 14, wherein the immunogenic fragment has a length of at least about 5 amino acids, or the variant has an at least about 80% identity to the P47 or an immunogenic fragment thereof.

20. A method of substantially blocking or substantially reducing transmission of a parasite of the Plasmodium genus in a population of humans comprising administering a pharmaceutical composition comprising P47 protein, an immunogenic fragment thereof, or a variant thereof, to at least one human.

21. The method of claim 20, wherein the parasite is P. falciparum and the P47 is Pfs47 (SEQ ID NO:1), or the parasite is P. vivax and the P47 is Pvs47 (SEQ ID NO:2).

22. The method of claim 20, wherein the composition further comprises one or more antigens of a human pathogen.

23. The method of claim 22, wherein the pathogen is selected from influenza, measles, mumps, diphtheria, tetanus, pertussis, poliovirus, hepatitis B virus, varicella, N. meningitides, and rubella.

24. (canceled)

25. The method of claim 20, wherein the immunogenic fragment has a length of at least about 5 amino acids, or the variant has at least about 80% identity to the P47 protein or an immunogenic fragment thereof.

26-27. (canceled)

28. A method of substantially blocking or substantially reducing transmission of a parasite of the Plasmodium genus in a population of humans comprising administering a pharmaceutical composition comprising antibodies or fragments thereof, specifically reactive to P47, or an immunogenic fragment or variant thereof, and a pharmaceutically acceptable carrier, to at least one human.

29. The method of claim 28, wherein the parasite is P. falciparum and the P47 protein is Pfs47 (SEQ ID NO:1), or the parasite is P. vivax and the P47 protein is Pvs47 (SEQ ID NO:2).

30. The method of claim 28, wherein the composition further comprises one or more antigens of a human pathogen.

31. The method of claim 30, wherein the pathogen is selected from influenza, measles, mumps, diphtheria, tetanus, pertussis, poliovirus, hepatitis B virus, varicella, N. meningitides, and rubella.

32. (canceled)

33. The method of claim 28, wherein the immunogenic fragment has a length of at least 5 amino acids, or the variant has at least about 80% identity to the P47 protein or an immunogenic fragment thereof.

34-68. (canceled)
